(19) United States
(12) Patent Application Publication
(10) Pub. No.: US 2009/0098057 A1
(43) Pub. Date: Apr. 16, 2009

(54) SILICA-CORED CARRIER PARTICLE

(76) Inventors: Shiying Zheng, Center Valley, PA (US); Lijun Dai, Rochester, NY (US); Ruizheng Wang, Rochester, NY (US); Tiecheng A. Qiao, Webster, NY (US); Wenyi Che, Rochester, NY (US); William J. Harrison, Pittsford, NY (US)

Correspondence Address: Susan L. Parulski, Patent Legal Staff;Carestream Health, Inc., 150 Verona Street, Rochester, NY, 14608, US

(21) Appl. No.: 11/872,866

(22) Filed: Oct. 16, 2007

(51) Int. Cl. A61K 49/00; A61B 5/00

(52) U.S. Cl. 424/9.6

(57) ABSTRACT

A nanoparticulate imaging probe with an oxide core, a biocompatible polymeric shell covalently attached to the oxide core, a dye, and a cleavable spacer that covalently binds the dye to the probe. When the spacer is cleaved, the dye is liberated from the probe. The emissions of the dye are quenched when the dye is bound to the probe and not quenched when the dye is liberated from the probe. The spacer can be, for example, a peptide. The oxide core can be, for example, a silicon oxide core.

### FIELD OF THE INVENTION

The present invention relates to an oxide-cored carrier particle having an exterior layer of functionalized polymer. In particular, this invention relates to nanoparticles for optical imaging.

### BACKGROUND OF THE INVENTION

Optically based biomedical imaging techniques, especially optical molecular imaging, are very powerful tools for studying the temporal and spatial dynamics of specific biomolecules and their interactions in real time in vivo and have been increasingly used to probe protein function and gene expression in vivo. Optical imaging techniques exhibit the great advantages of high temporal (picosecond, important in functional imaging) and spatial (submicron, important in in vivo microscopy) resolutions, high sensitivity (single molecule level) and minimal invasion. They also offer the potential for simultaneous use of multiple and distinguishable probes (important in molecular imaging) and safety (no ionizing radiation). These techniques have advanced over the past decade due to rapid developments in laser technology, sophisticated reconstruction algorithms and imaging software originally developed for non-optical, tomographic imaging modes such as CT and MRI.

Of the various optical imaging techniques investigated to date, near-infrared (NIR, 700 to 1000 nm wavelength) fluorescence (NIRF) imaging is of particular interest for non-invasive in vivo imaging because of the relatively low tissue absorbance, minimal autofluorescence of NIR light, and deep tissue penetration of up to 6-8 centimeters. In near infrared fluorescence imaging, filtered light or a laser with a defined bandwidth is used as a source of excitation light. The excitation light travels through body tissues. When it encounters a near infrared fluorescent molecule (“contrast agent or probe”), the excitation light is absorbed. The fluorescent molecule then emits light (fluorescence) spectrally distinguishable (slightly longer wavelength) from the excitation light. Despite good penetration of biological tissues by near infrared light, conventional near infrared fluorescence probes are subject to many of the same limitations encountered with other contrast agents, including low signal/noise ratios.

A number of NIRF contrast-enhanced optical imaging probes have been developed and evaluated in small animals. These studies have established the use of NIR optical imaging in diagnosis, molecular characterization, and monitoring of treatment response in a number of disease models. Successful translation of NIRF optical imaging into clinical use requires advances on several fronts, including development of tomographic optical imaging systems capable of imaging signals in deep organs in vivo, development of endoscopes, laparoscopes, and other intraoperative imaging devices to sense fluorophores at body surfaces, and particularly, the development and validation of fluorescence-based contrast agents or probe.

Nanoparticles have been increasingly used in a wide range of biomedical applications such as drug carriers and imaging agents. They are engineered materials with dimensions typically smaller than 100 nm, small enough to reach almost anywhere in the body and can be easily derivatized with a variety of targeting ligands, multiple imaging moieties for multiple modalities imaging, or loaded with multiple molecules of a contrast agent, providing a significant boost in signal intensity for diverse imaging modalities. NIRF imaging based on nanoparticulate imaging probes is rapidly emerging as an advanced technology for noninvasive cancer detection, diagnostic and therapeutic applications. Nanoparticle-based imaging probe offers potential advantages over small molecule or low molecular weight polymer-based probe such as longcirculating time for effective tumor delivery because small probes are subjected to fast excretion in vivo, giventrenal clearance of small molecules and reticuloendothelial system clearance of non-immunologtically shielded compounds. Several reports have featured quantum dots (QDs) (Warren, C. W. et al. Science 1998, 281, 2016-2018) composed of a fluorescent core encapsulated within novel polymeric or lipid-based layers for NIRF optical imaging in cancer imaging in animals. However, most QDs are made of toxic material such as cadmium, and it has yet been established that QDs are sufficiently stable to avoid becoming toxic in the body. The design and synthesis of smart nanoprobes is an enabler for NIRF imaging to be successful.

More recently, there has been intense interest focused upon developing surface-modified nanoparticulate materials that are capable of carrying biological, pharmaceutical or diagnostic components. The components, which might include drugs, therapeutics, diagnostics, and targeting moieties can then be delivered directly to diseased tissue or bones and be released in close proximity to the diseased tissue and reduce the risk of side effects to the patient. This approach has promised to significantly improve the treatment of cancers and other life threatening diseases and may revolutionize their clinical diagnosis and treatment. The components that may be carried by the nanoparticles can be attached to the nanoparticle by well-known bio-conjugation techniques; discussed at length in Bioconjugate Techniques, G. T. Hermanson, Academic Press, San Diego, Calif. (1996). The most common bio-conjugation technique involves conjugation, or linking, to an amine functionality.

Certain nanoparticles were recently proposed as carriers for certain pharmaceutical agents. See, e.g., Sharma et al. Oncology Research 8, 281 (1996); Zobel et al. Antisense Nucl. Acid Drug Dev., 7:483 (1997); de Verdiere et al. Br. J. Cancer 76, 198 (1997); Hussein et al., Pharm. Res., 14, 613 (1997); Alyautdin et al. Pharm. Res. 14, 325 (1997); Hrkach et al., Biomaterials, 18, 27 (1997); Torchilin, J. Microencapsulation 15, 1 (1988); and literature cited therein. The nanoparticle chemistries provide for a wide spectrum of rigid polymer structures, which are suitable for the encapsulation of drugs, drug delivery and controlled release. Some major problems of these carriers include aggregation, colloidal instability under physiological conditions, low loading capacity, restricted control of the drug release kinetics, and synthetic preparations which are tedious and afford very low yields of product.

Many authors have described the difficulty of making colloidally stable dispersions of colloids having surface modified particles. Achieving colloidal stability under physiological conditions (pH 7.4 and 137 mM NaCl) is yet even more difficult. Burke and Barret (Langmuir, 19, 3297(2003)) describe the adsorption of the amine-containing polyelectrolyte, polyallylamine hydrochloride, onto 70-100 nm silica particles in the presence of salt. The authors state (p. 3299) “the concentration of NaCl in the colloidal solutions was maintained at 1.0 mM because higher salt concentrations lead to flocculation of the colloidal suspension.”

Colloidal silica particles have been developed for various applications especially surface modified silica particles such as silica core and polymer shell nanocomposite materials in high-tech applications including chemical and biochemical sensors, display devices, memory storage media and micromechanical devices. The following patents disclose various methods of preparation of core-shell nanoparticles and their utilities. However, none of these disclose the utility of nanoparticles as carriers for imaging probes.

U.S. Pat. No. 6,592,847 (Weissleder et al.) entitled “Intramolecularly-quenched near infrared fluorescent probes” discloses an activatable near-infrared fluorescence (NIRF) probe using a polymer as a carrier. However, the near-infrared fluorescence is not completely quenched and the signal/noise ratios are not optimal.

U.S. Pat. No. 7,033,524, issued Apr. 25, 2006, entitled “Polymer-based nanocomposite materials and methods of production thereof” discloses the methods of producing polymer-based nanoparticles via emulsion polymerization techniques to generate composite materials. The core materials include polymer or inorganic based oxide and the core was coated with a layer of polymer as a shell. However, there is no chemical bonding between cores and shells and there is no sufficient adhesion between the cores and shells which creates much difficulties during the particle making process. The size of the nanoparticles disclosed are in the range of 100 mn to 1 micron.

U.S. Pat. No. 6,881,804, issued Apr. 19, 2005, entitled “Porous, molecularly imprinted polymer and a process for the preparation thereof” describes a porous, molecularly imprinted polymer and a process for its preparation. The porous silica particles were used to fill the monomers in the pores for polymerization and the silica template was removed after polymerization to create the porous structure. The silica particles were used as templates.

U.S. Pat. No. 6,720,007, issued Apr. 13, 2004, entitled “Polymeric microspheres” discloses a method of preparation of hollow polymeric microspheres using silica particles as sacrificial templates. Polymers were grafted onto the surface of silica particles via surface initiated polymerization and then the silica particles were etched off to leave the hollow spheres.

U.S. Pat. No. 6,627,314, issued Sep. 30, 2003, entitled “Preparation of nanocomposite structures by controlled polymerization” describes preparation of nanocomposite particles and structures by surface initiated polymerization from functional inorganic colloidal silica nanoparticles. However, the patent does not disclose any utility of such nanocomposite materials. It is well accepted that colloidal particles can exhibit preferential tumor accumulation after their systemic administration because of the enhanced permeability and retention (EPR) effect, which is characterized by microvascular hyperpermeability to circulating colloidal particles and impaired lymphatic drainage in tumor tissues. This passive manner of delivery without specific binding to cellular targets (i.e., passive targeting) can be highly effective for water-soluble macromolecules and polymeric micelles. It has been recognized that the tumor accumulation of colloidal particles based on the enhanced permeability and retention (EPR) effect can only be successful when they possess a prolonged blood circulation time. A number of factors, such as size, size distribution, composition, and surface hydrophilicity, can influence the circulation of nanoparticles in the blood. In particular, surface modification with flexible, hydrophilic poly(ethylene glycol) (PEG) has proven to be effective in preventing the uptake of various polymer-based nanoparticles by the macrophages of the mononuclear phagocytic system (MPS).

There remains a need for an activatable imaging probe with improved signal to noise ratio.

### SUMMARY OF THE INVENTION

The present invention relates to a nanoparticle-based imaging probe comprising a core-shell nanoparticle and at least one dye, wherein the core-shell nanoparticle comprise an oxide core and polymer shell, wherein the oxide core is silica or other metal oxide, and the polymer shell comprises biocompatible polymers and reactive functional groups, and the dye is immobilized in the core or the polymer shell. The imaging probe emits substantial fluorescence only after activation, i.e. interaction with a target enzyme or tissue.

### ADVANTAGEOUS EFFECT OF THE INVENTION

The present invention includes several advantages, all of which may or may not be incorporated in a single embodiment.

The nanoparticle-based imaging probe of the present invention provides improved signal/noise ratios. The nanoparticles of the imaging probe of the present invention provide a carrier for biological, pharmaceutical or diagnostic components. In the present invention polymer-grafted shell and silica-cored nanoparticles are used as carriers for the activatable imaging probe. The polymer shell contains primary amine functional groups and PEG. Because of the tremendous surface area introduced through nanoparticles, the nanoparticles of the present invention allow for the attachment of enough PEG molecules to reduce immunological response, are stable within a broad window of conditions and offer high biological compatibility. Furthermore, they provide high loading levels of dyes to achieve higher signal amplification.

### BRIEF DESCRIPTION OF THE DRAWINGS

FIG. 1 is an illustration of a pathway used to construct one self-quenching probe of the invention; FIGS. 2A, 2B, and 2C are illustrations of a schemes to generate a FRET probe for use with the present invention.

FIG. 3 is a depiction of one structure of one peptide linker for use with the instant invention.

FIG. 4 is a schematic diagram of the synthesis of one core-shelled nanoparticle of the invention.

FIG. 5, which includes FIGS. 5A and 5B, shows the activation of a probe by incorporating MMP-2-specific peptide sequence via self-quenching.

FIG. 6 shows the activation of a probe by incorporating MMP-2-specific peptide sequence via FRET.

FIG. 7 and FIG. 8 are a series of NIR and phase contrast images of the invention.

### DETAILED DESCRIPTION OF THE INVENTION

The present invention relates to a nanoparticle-based imaging probe comprising a core-shell nanoparticle and at least one dye (including fluorescent dye and quencher), wherein said core-shell nanoparticle comprise an oxide core and polymer shell, wherein said oxide core is silica or other metal oxide, and said polymer shell comprising biocompatible segments and reactive functional groups, and said dye is immobilized in the core or the polymer shell. The imaging probe emits substantial fluorescence only after activation, i.e. interaction with a target enzyme or tissue. This increases the signal/noise ratio by several orders of magnitude and enables non-invasive, near infrared fluorescence imaging of internal target tissues in vivo, based on enzymatic activity present in the target tissue. Accordingly, the invention features a fluorescence-quenched probe comprising a core-shell nanoparticle and a plurality of near infrared dyes. The core-shell nanoparticle comprises an oxide core such as silica oxide or metal oxide such as aluminum oxide, iron oxide, zinc oxide or zirconium oxide, and a polymer shell. A plurality of near infrared dye molecules are covalently linked to the core of the oxide core of the nanoparticle or at the polymer shell. The fluorescence-quenching is caused by self-quenching of the near infrared fluorophore or by energy transfer from the near infrared fluorophore to a quencher. Fluorescence activation is induced by enzymatic cleavage at fluorescence activation sites.

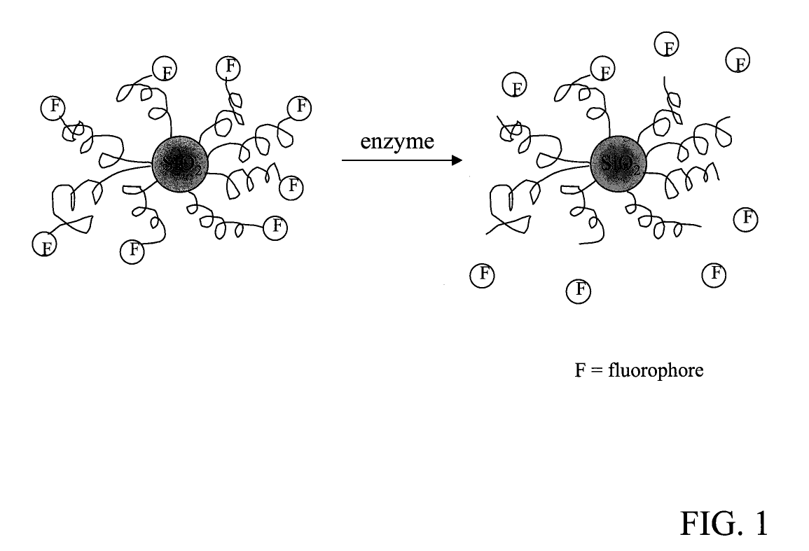

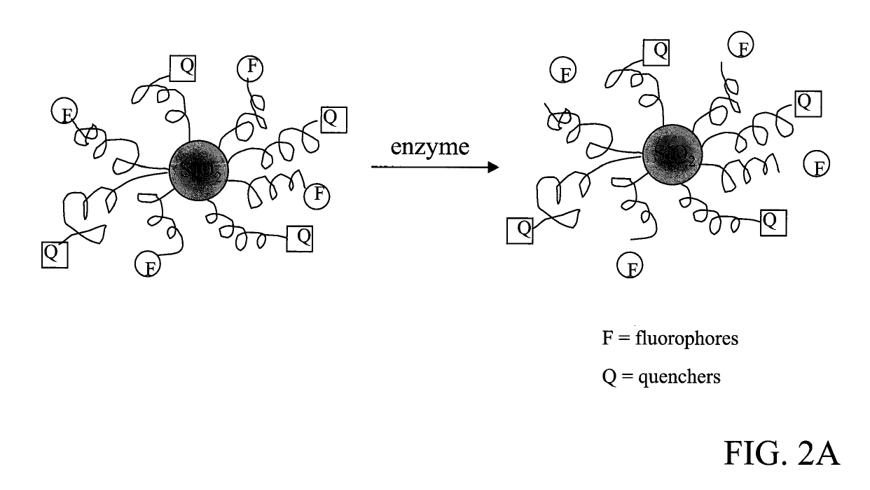

The activation schemes are illustrated in FIG. 1, FIG. 2A, FIG. 2B, and FIG. 2C. The fluorescent dyes are attached to the polymer shell via enzyme-specific peptide spacer groups. By self-quenching strategy in FIG. 1, the fluorophore in the polymer shell of the imaging probe starts to quench each other because of close proximity. Upon enzymatic cleavage (such as by MMP-2) of the peptide linker, the fluorophores, along with peptide fragment, are released from the imaging probe leading to a de-quenched state. By fluorescence resonance energy transfer (FRET) strategy in FIG. 2A: the fluorophores are attached to the polymer shell of the nanoparticle via peptide spacer groups; and quencher dyes are directly attached to the polymer shell of the nanoparticles, the quenchers absorb part of the emission from the fluorophores. After enzymatic cleavage of the peptide linker, fluorphores, along with peptide fragments, are released leading to significant fluorescence increase. FIGS. 2B and 2C illustrate the FRET strategy by incorporating dye either fluorophore or quencher into the core of the nanoparticle.

The core of the core-shell nanoparticle can be any oxide, such as silica oxide, or metal oxide, such as aluminum oxide, iron oxide, zinc oxide or zirconium oxide. Preferably, the core is silica oxide or iron oxide, and most preferably, the core is silica oxide.

The shell of the nanoparticle includes a biocompatible polymer, and reactive functional groups. For example, the polymer can be a polypeptide, a polysaccharide, a nucleic acid, or a synthetic polymer. Useful polypeptides include, for example, polylysine, albumins, and antibodies. The polymers also can be a synthetic polymer such as poly(alkylene oxides) for example poly(ethylene oxide), poly(2-ethyloxazolines), poly(saccharides), dextrans and vinyl polymers containing poly(ethylene oxide)poly(ethylene oxide) moiety. Preferably hydrophilic components are poly(ethylene oxide) and vinyl polymers containing poly(ethylene oxide) moiety, and more preferably poly(ethylene oxide) poly(meth(acrylates)) containing poly(ethylene oxide) moiety, polystyrenes containing poly(ethylene oxide) moiety, poly(meth(acrymides) containing poly(ethylene oxide) moiety, polyglycolic acid, polylactic acid, poly(glycolic-colactic) acid, polydioxanone, polyvalerolactone, poly(ε-caprolactone), poly(3-hydroxybutyrate), poly(3-hydroxyvalerate)polytartronic acid, and poly(β-malonic acid). The reactive functional groups include, but are not limited to, thiols, chloromethyl, bromomethyl, amines, carboxylic acid or activated ester, vinylsulfonyls, aldehydes, epoxies, hydrazides, succinimidyl esters, maleimides, a-halo carbonyl moieties (such as iodoacetyls), isocyanates, isothiocyanates, 4-fluoro-5-nitro-benzoate, and aziridines. Preferably the reactive functional group is a thiol, a carboxylic acid, an amine, 4-fluoro-5-nitro-benzoate, or a carboxylic acid activated ester. More preferably, the reactive functional group is an amine, 4-fluoro-5-nitro-benzoate, or an activated carboxylic acid ester.

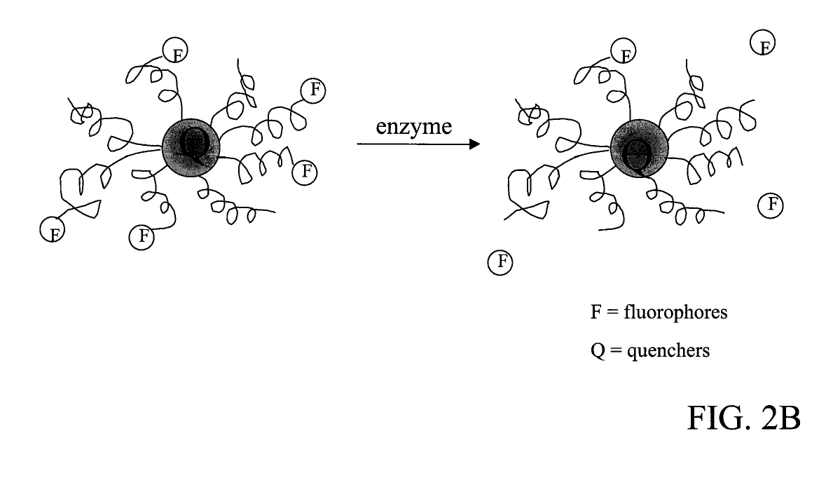

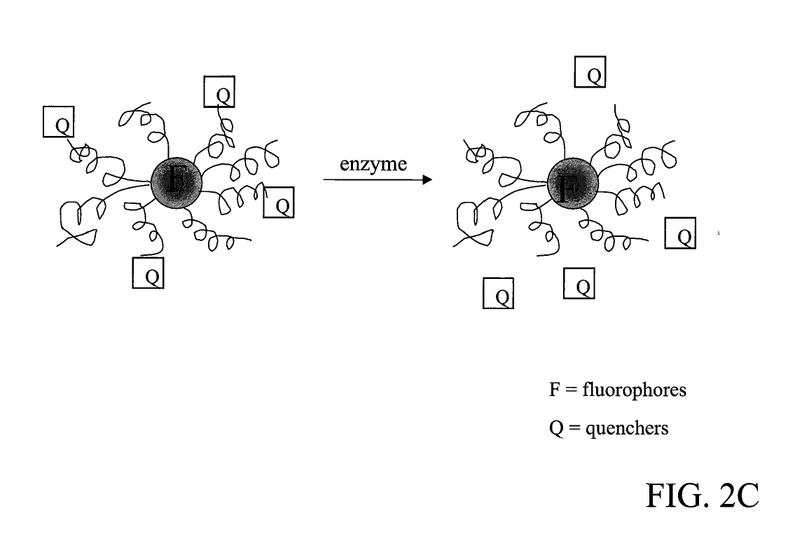

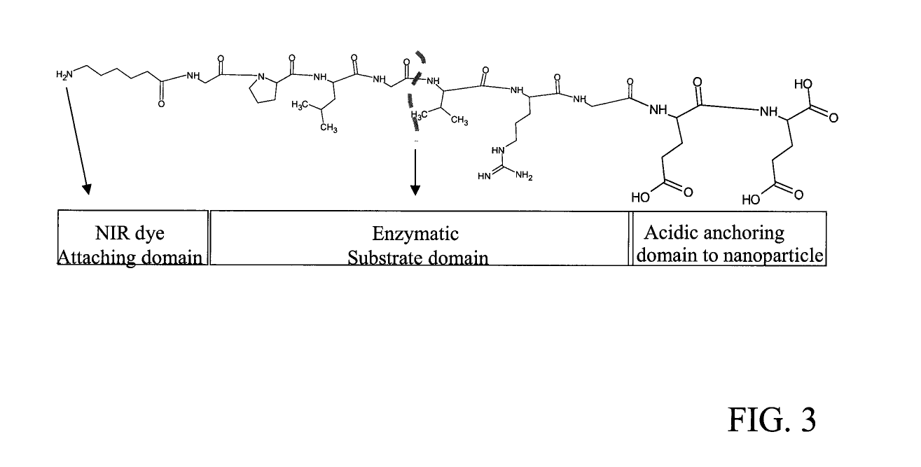

Fluorescence activation sites can be located in the shell of the nanoparticle, e.g., when the near infrared dye and/or quencher can be linked to the polymer shell by a spacer containing a fluorescence activation site. The spacers can be oligopeptides. Oligopeptide sequences useful as spacers include sequences such as those disclosed in International Publication No. WO2004/026344. FIG. 3 the construct of a peptide sequence with anchoring domains to attach dyes and to attach to the polymer shell of the nanoparticle.

Near infrared fluorescent dyes useful in this invention include Cy5.5, Cy5, Cy7, IRD41, IRD700, NIR-1, LaJolla Blue, indocyanine green (ICG) and analogs thereof, indotricarbocyanine (ITC), and chelated lanthanide compounds that display near infrared fluorescence. The fluorescent dyes can be covalently linked to the polymer shell of the nanoparticle, or spacers, using any suitable reactive group on the fluorescent dyes and a compatible functional group on the polymer shell or spacer. A probe according to this invention also can include a targeting moiety such as an antibody, antigen-binding antibody fragment, a receptor-binding polypeptide, or a receptor-binding polysaccharide.

The invention also features an in vivo optical imaging method. The method includes: (a) administering to a living animal or human a fluorescence-quenched probe comprising fluorescence activation sites by enzymatic cleavage that accumulates preferentially in a target tissue; (b) allowing time for (1) the probe to accumulate preferentially in the target tissue, and (2) enzymes in the target tissue to activate the probe by enzymatic cleavage at fluorescence activation sites, if the target tissue is present; (c) illuminating the target tissue with near infrared light of a wavelength absorbable by the fluorescent dyes; and (d) detecting fluorescence emitted by the fluorescent dyes.

The above method can be used, e.g., for in vivo imaging of a tumor in a human patient, or in vivo detection or evaluation of arthritis in a joint of a human patient. The invention also features an in vivo method for selectively imaging two different cell or tissue types simultaneously. The method includes administering, to an animal or human patient, two different fluorescence-quenched probes, each of which accumulates preferentially in a target tissue. Each of the two probes includes fluorescence activation sites by enzymatic cleavage and each of the two probes comprises a fluorescent dye whose fluorescence wavelength is distinguishable from that of the other fluorescent dye, and each of the two probes contains a different activation site.

Whenever used in the specification the terms set forth shall have the following meaning:

The term “fluorescence activation site” means a covalent bond within a probe, which bond is: (1) cleavable by an enzyme present in a target tissue, and (2) located so that its cleavage liberates a fluorescent dye or a quencher from being held in a fluorescence-quenching position.

The term “fluorescence-quenched” means fluorescent dyes or fluorescent dyes and quencher are covalently linked (directly or indirectly through a spacer) the polymer shell so that the fluorescent dyes or fluorescent dyes and quenchers are maintained in a position relative to each other that permits them to interact photochemically and quench the fluorescence.

The term “targeting moiety” means a moiety bound covalently or non-covalently to a fluorescence-quenched probe, which moiety enhances the concentration of the probe in a target tissue relative to surrounding tissue. Unless otherwise defined, all technical and scientific terms used herein have the same meaning as commonly understood by one of ordinary skill in the art to which this invention belongs.

The term nanoparticle or nanoparticulate refers to a particle with a size of less than 100 nm.

The term “colloid” refers to a mixture of small particulates dispersed in a liquid, such as water. The term “biocompatible” means that a composition does not disrupt the normal function of the bio-system into which it is introduced. Typically, a biocompatible composition will be compatible with blood and does not otherwise cause an adverse reaction in the body. For example, to be biocompatible, the material should not be toxic, immunogenic or thrombogenic.

The term “biodegradable” means that the material can be degraded, either enzymatically or hydrolytically, under physiological conditions to smaller molecules that can be eliminated from the body through normal processes.

The “stable dispersion” means that the solid particulates do not aggregate, as determined by particle size measurement, and settle from the dispersion, usually for a period of hours, preferably weeks to months. Terms describing instability include aggregation, agglomeration, flocculation, gelation and settling. Significant growth of mean particle size to diameters greater than about three times the core diameter, and visible settling of the dispersion within one day of its preparation is indicative of an unstable dispersion.

The term “swollen” refers to the solvated state which the polymer associates with the solvent molecules rather than with each other, thereby expanding the total volume occupied by the single polymer molecule.

The term “water compatible” refers to a material which exists in a swollen state in water over the temperature range of 5-80° C.

The nanoparticle is stable in solution or dispersion. The dispersion is said to be stable if the solid particulates do not aggregate, as determined by particle size measurement, and settle from the dispersion, usually for a period of hours, preferably weeks to months. Terms describing instability include aggregation, agglomeration, flocculation, gelation and settling. Significant growth of mean particle size to diameters greater than about three times the core diameter, and visible settling of the dispersion within one day of its preparation is indicative of an unstable dispersion. Preferably the nanoparticle is stable at 20-35° C. in 0.137M NaCl at pH 7.4. Most preferably the nanoparticle is stable in 0.8 M NaCl.

The nanoparticle is comprised of a silica core, a polymer shell and at least one dye. The polymer shell is covalently attached to the silica core and includes a plurality of reactive functional groups. The dye can be immobilized in the silica core or in the polymer shell. When attached to the polymer shell, the dye can be directly or indirectly attached via a spacer covalently bound to the polymer shell. Degradation of the spacer causes the dye to be released from the nanoparticle. Other agents such as a therapeutic agent, a targeting agent, or a diagnostic agent, can be attached to the nanoparticle, directly or indirectly, via a spacer. In one embodiment, the spacer group is a polypeptide and the degradation is an enzyme catalyzed cleavage. In another embodiment, the spacer is degraded hydrolytically.

The polymer shell can be a homopolymer or a copolymer, and have a weight average molecular weight of from 1,000 to 1,000,000, and preferably from 2,000 to 100,000, and more preferably from 3,000 to 80,000 as measured by static light scattering or by size exclusion chromatography.

Preferably, the nanoparticles have a diameter of from 1 nm to 1000 nm, and more preferably from 5 nm to 200 nm, and most preferably from 10 nm to 100 mn. The particle size(s) of the nanoparticle may be characterized by a number of methods, or combination of methods, including, light-scattering methods, sedimentation methods such as analytical ultracentrifugation, hydrodynamic separation methods such as field flow fractionation and size exclusion chromatography, and electron microscopy. The nanoparticles in the examples were characterized primarily using light-scattering methods. Light-scattering methods can be used to obtain information regarding volume median particle diameter, the particle size number and volume distribution of nanoparticles, standard deviation of the distribution(s) and the distribution width.

In a preferred embodiment, the core is silica and silica particles can be prepared by the Stober process wherein a tetraorthosilicate is controllably hydrolysed and self-condenses to form particles. The size of the particle produced by the Stober process is tunable between the ranges 10-1000 nm in dispersions of ethanol, other polar solvents, or in aqueous basic solutions such as ammonium hydroxide. Stober particles, as particles produced by the Stober process are known, in alcohol dispersion or alcohols, are kinetically stabilized by electrostatic forces, generated by negative charges from ionized surface silanol groups. Thermodynamically stable particles may be prepared by condensation of these surface silanol groups on the Stober particles with a monoalkoxysilane. If the monoalkoxysilane incorporate additional functionality attached to, for example, the alkoxy group, the condensation reaction will incorporate functional groups onto the particle surface to produce, for example, a polymerization initiation site.

The particles may be produced with narrow particle size distributions and a certain amount of functional groups attached to the particle surface. The number of functional groups incorporated on the particle may be controlled by the mole ratio of initiator functional silane to non-functional silane used in the process as well as by other methods known to one skilled in the art.

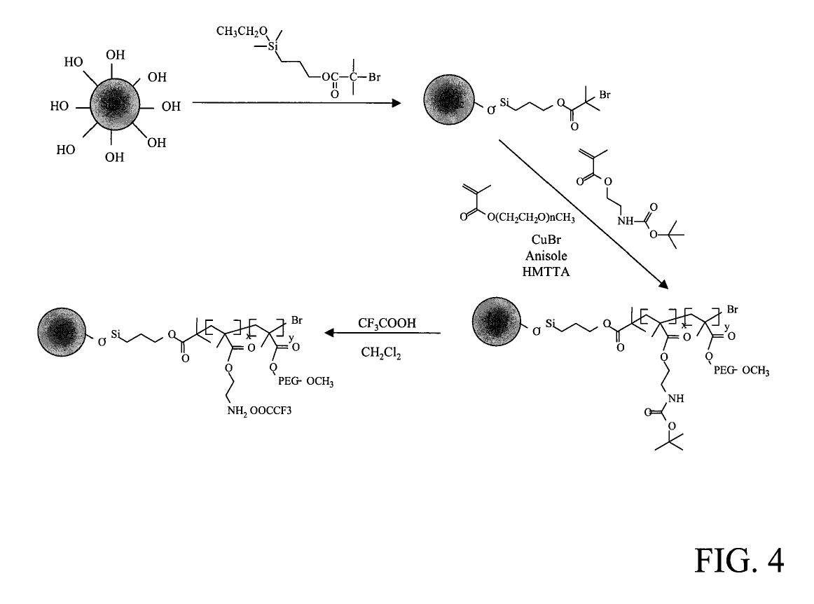

Alternatively, the amount of functional groups may be controlled by direct addition of a functional monoalkoxysilane, such as (3-(2-bromoisobutyryloxy)propyldimethyethoxysilane), to silica particle surface. After the particle surface is functionalized to the desired degree, an excess of hexamethyldisilazane may then be added to consume any remaining residual silanol groups. Stable, dispersible particles containing an attached functional group capable of initiating a polymerization reaction may then be isolated. Such particles are referred to as inorganic colloidal initiator particles. Characterization of such surface functional Stober particles can be conducted by elemental analysis, dynamic light scattering (DLS) and atomic force microscope prior to use of the particles as inorganic colloidal initiators. FIG. 4 illustrates the multi-step synthesis of the inorganic colloidal silica initiators and subsequent polymerization by ATRP.

The synthesis of the inorganic colloidal initiator nanoparticles may be conducted in a solvent such as tetrahydrofuran (THF), methyl ethyl ketone or dioxane. The initiator particles produced by this process were capable of being isolated and, subsequently, redispersed. It may be desirable to conduct only a partial initial surface treating reaction with a surface-treating agent comprising the desired functionality to provide particles with remaining residual reactive surface sites. As used herein, a surface treating agent is a molecule, such as a monoalkoxysilane, which will react with the particle surface. The surface-treating agent may incorporate desired functionality or be used to stabilize the particle surface. In the examples described later, substantially uniform particles with diameters between 15-20 nm and 1000 initiation sites on the surface were prepared. The number of initiating sites can be varied by varying the ratio of the surface treating agents and could vary from an average of 1 to 1,000,000 or more depending on particle size and initiation site density. Exemplary particles with 300 to 3000 initiating sites were prepared, however this range can be expanded using the methods described herein if desired. It is expected that the preferred number of functional groups on each particle would be in the range of 100 to 100,000, and more preferably in the range of 300 to 30,000 to produce the advantageous properties of the nanocomposite particles. Control over the number of initiating sites on a particle allows one to control the graft density of the attached polymer chains and thereby the packing density of the polymer chains as polymer shell. A high density of initiating sites provides for maximum incorporation of grafted polymer chains and tethered chains that are in an extended, brush-like state. Whereas a loose packing density can be employed to provide tethered chains that may assume a coiled conformation at higher molar mass. Such a coiled chain formation may be employed when one wishes to use the first attached block copolymer as a medium for further incorporation of occluded materials such as drugs or cosmetics for subsequent controlled delivery.

In a preferred embodiment, the inorganic colloidal initiator particles comprise a nanoparticle and a functional group having an initiation site capable of initiating a free radical polymerization process. More specifically a controlled free radical polymerization initiator, such as those for atom transfer radical polymerization (ATRP), nitroxide mediated polymerization (Husseman, M. et al. Macromolecules 1999, 32, 1424-1431) or reversible addition-fragmentation chain transfer polymerization (RAFT) (Li, C. et al. Macromolecules, 2005, 38, 5929) was introduced to the surface of the silica particle. Preferably, atom transfer radical polymerization (ATRP) initiator bromo-isobutyrate was introduced to the surface by reacting with 3-(2-bromoisobutyryloxy)propyldimethyethoxysilane. More specifically, the functional group comprises an initiator moiety having a radically transferable atom or group that participates in a controlled or living free radical polymerization such as atom transfer radical polymerization (ATRP). Preferred atom transfer radical polymerization (ATRP) initiator includes phenyl ethyl chloride, phenyl ethyl bromide, phenyl sulfonyl chloride, and 2-bromoethylisobutyrate. The polymerization process may be catalyzed by a transition metal complex which participates in a reversible redox cycle with at least one of the group and a compound having a radically transferable atom or group, to form a nanocomposite particle with a tethered or grafted polymer chain as polymer shell. The present invention may include further polymerization of additional radically polymerizable comonomers on the tethered polymer chain to form a tethered copolymer chain. The particle may be silicon based including, for example, silica, silicates and polysilsesquioxane.

Controlled/living radical polymerization has been explored as a means of producing well-defined polymers. Atom transfer radical polymerization (ATRP) involves the use of a novel initiating systems. The initiation system is based on the reversible formation of growing radicals in a redox reaction between various transition metal compounds and an initiator, for example alkyl halides, aralkyl halides or haloaklyl esters.

Atom transfer radical polymerization (ARTP) (Wang, J.-S.; Matyjaszewski, K. J. Am. Chem. Soc. 1995, 117, 5614-5615; Matyjaszewski, K. J.; Wang, J.-S. U.S. Pat. No. 5,763,548) has great synthetic power to control the molecular architecture of polymers and is exceptionally robust method of producing block or graft copolymers. It offers several advantages over other polymerization routes including control over molecular weight and molecular weight distribution, and the polymers can be end-functionalized or block copolymerization upon the addition of other monomers. Atom transfer radical polymerization (ATRP) is one of the most successful methods to polymerize styrenes, (meth)acrylates and a variety of other monomers in a controlled fashion, yielding polymers with molecular weights predetermined by the ratio of the concentrations of consumed monomer to introduced initiator and with low polydispersities. Because of its radical nature, atom transfer radical polymerization is tolerant to many functionalities in monomers leading to polymers with functionalities along the chains. With atom transfer radical polymerization, functionality and architecture can be combined resulting in multifunctional polymers of different compositions and shapes such as block copolymers, multi-armed stars or hyperbranched polymers.

In another embodiment, the inorganic colloidal initiator particles comprise a nanoparticle and a functional group having an initiation site capable of initiating a ring opening polymerization of cylic oxide and N-carboxyanhydride (NCA) of amino acids. More specifically the initiation site containing amine or hydroxyl groups.

The polymer shell is tethered or grafted onto the inorganic colloidal initiator nanoparticles via surfaced initiated or surface confined atom transfer radical polymerization (ATRP). Surface initiated or surfaced confined atom transfer radical polymerization (ATRP) is simple, flexible and enables control over the shell thickness and composition of the poloymer shell by adjusting polymerization time and monomer concentration. A wide range of monomers are useful for the preparation of polymer shell. The monomers include, but are not limited to, styrenes, (meth)acrylates, and (meth)acrylamide. For ring opening polymerization, the monomers include natural or synthetic N-carboxyanhydride (NCA) of amino acids, and cyclic oxide such as ethylene oxide or propylene oxide.

Reactive functional groups are incorporated into the polymer shell by either employing monomers with the functional groups or modifying polymer shell by chemical reactions after the shell is formed. The reactive functional groups include but are not limited to thiols, chloromethyl, bromomethyl, amines, carboxylic acid or activated ester, vinylsulfonyls, aldehydes, epoxies, hydrazides, succinimidyl esters, maleimides, a-halo carbonyl moieties (such as iodoacetyls), isocyanates, isothiocyanates, and aziridines. Preferably the reactive functional group is a thiol, a carboxylic acid, an amine, 4-fluoro-5-nitro-benzoate, or a carboxylic acid activated ester. More preferably, the reactive functional group is an amine, 4-fluoro-5-nitro-benzoate, or a carboxylic acid activated ester.

To assemble the biological, pharmaceutical or diagnostic components to a described nanoparticle used as a carrier, the components can be associated with the nanoparticle carrier through a linkage. By “associated with”, it is meant that the component is carried by the nanoparticle. The component can also be dissolved and incorporated in the nanoparticle non-covalently.

Generally, any manner of forming a linkage between a biological, pharmaceutical or diagnostic component of interest and a nanoparticle used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanoparticle used as a carrier through the formation of amide, ester or imino bonds between acid, aldehyde, hydroxy, amino, or hydrazo groups on the respective components of the complex. Art-recognized biologically labile covalent linkages such as imino bonds and so-called “active” esters having the linkage —O—O— or —COOCH are preferred. The biological, pharmaceutical or diagnostic component of interest may be attached to the polymer shell after it is formed or alternately the component of interest may be pre-attached to a polymerizable unit and polymerized directly into the polymer shell during the its preparation. Hydrogen bonding, e.g., that occurring between complementary strands of nucleic acids, can also be used for linkage formation.

In a preferred embodiment of this invention, the biological, pharmaceutical or diagnostic component of interest is attached to the polymer shell by reaction with the reactive functional group on the polymer shell. Preferably this reactive functional group on polymer shell is a carboxylic acid, an amine, a 4-fluoro-5-nitro-benzoate, or a carboxylic acid activated ester. Most preferably, this attachment occurs via a linking polymer.

The linking polymer may be used in both the acylation and alkylation approaches and is compatible with aqueous and organic solvent systems, so that there is more flexibility in reacting with useful groups and the desired products are more stable in an aqueous environment, such as a physiological environment. In one embodiment, the linking polymer has a poly(ethylene glycol) backbone structure which contains at least two reactive groups, one at each end. The poly(ethylene glycol)macromonomer backbone contains a radical polymerizeable group at one end. This group can be, but is not necessarily limited to a methacrylate, acrylate, acrylamide, methacrylamide, styrenic, allyl, vinyl, maleimide, or maleate ester. The poly(ethylene glycol)macromonomer backbone additionally contains a reactive chemical functionality at the other end which can serve as an attachment point for other chemical units, such as quenchers or antibodies. This chemical functionality may be, but is not limited to thiols, carboxylic acids, primary or secondary amines, vinylsulfonyls, aldehydes, epoxies, hydrazides, succinimidyl esters, maleimides, a-halo carbonyl moieties (such as iodoacetyls), isocyanates, isothiocyanates, and aziridines. Preferably, these functionalities will be carboxylic acids, primary amines, maleimides, vinylsulfonyls, or secondary amines. Most preferably, one of the reactive groups is an acrylate, cyanoacrylate, or a methacrylate which is useful for forming polymer shell and reacting with thiols through Michael addition. The other reactive group is useful for conjugation to contrast agents, dyes, proteins, amino acids, peptides, antibodies, bioligands, therapeutic agents and enzyme inhibitors. The linking polymer may be branched or unbranched. Preferably, for therapeutic use of the end-product preparation, the linking polymer will be pharmaceutically acceptable. The poly(ethylene glycol)macromonomer may have a molecular weight of between 300 and 10,000, preferably between 500 and 5000.

A particularly preferred water-soluble linking polymer for use herein is a poly(ethylene glycol) derivative of Formula I. The poly(ethylene glycol) (PEG) backbone of the linking polymer is a hydrophilic, biocompatible and non-toxic polymer of general formula H(OCH(2)CH(2))(n)OH, wherein n>4.

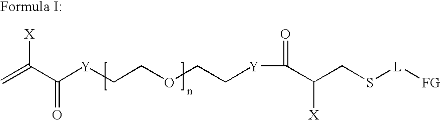

wherein X═CH₃ or H, Y═O, NR, or S, L is a linking group or spacer, FG is a functional group, n is greater than 4 and less than 1000. Most preferably, X═CH₃, Y═O, NR, L is alkyl or aryl and FG is 4-fluoro-5-nitrobenzoate, NH₂ or COOH, and n is between 6 and 500 or between 10 and 200. Most preferably, n=16. 4-fluoro-5-nitrobenzoate is a useful moiety to attach any component with amine groups (Ladd, D. L. et al. Analytical Biochemistry (1993, 210, 258-261)).

The following is a list of preferred monomers to form linking polymers, but is not intended to an exhaustive and complete list of all linking polymers according to the present invention:

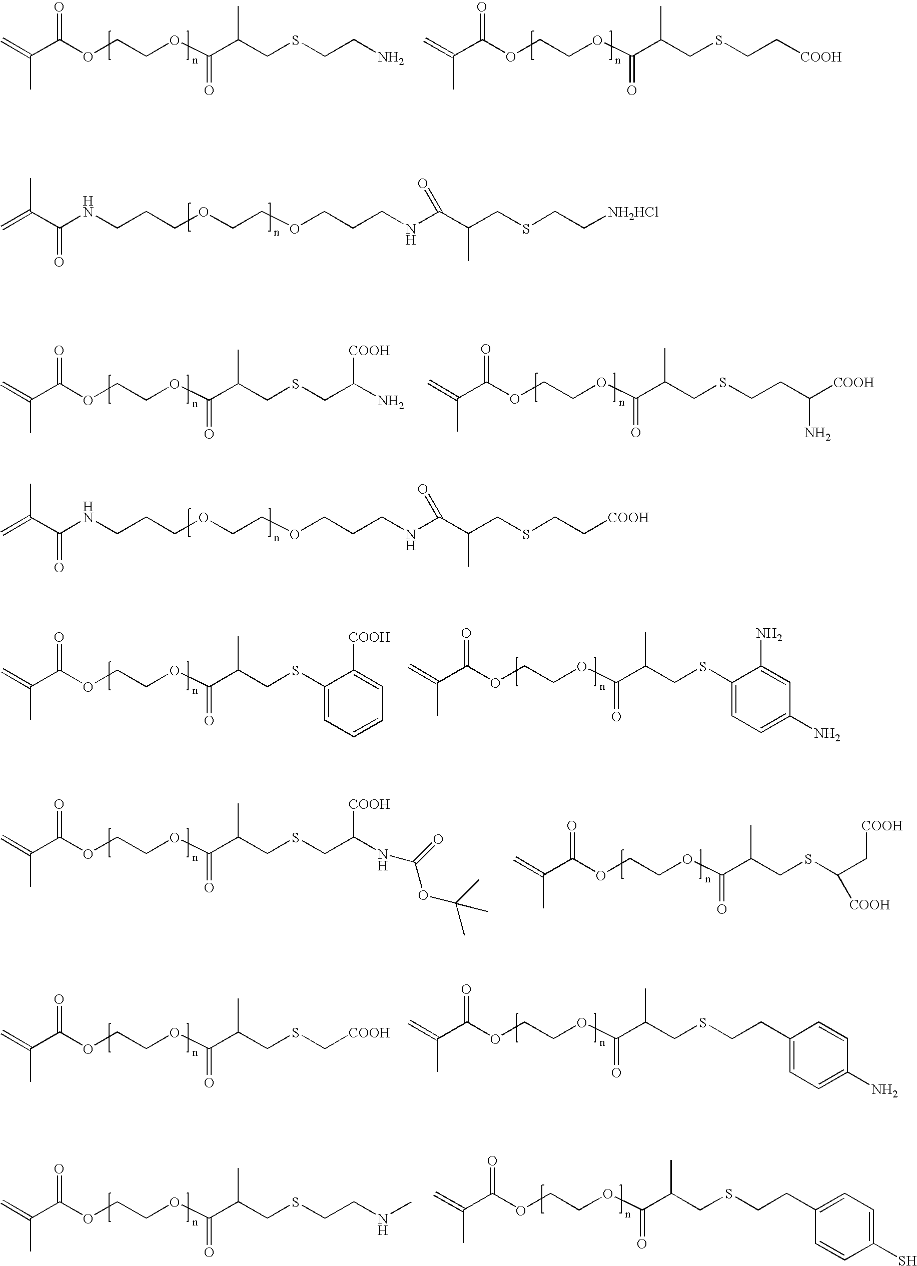

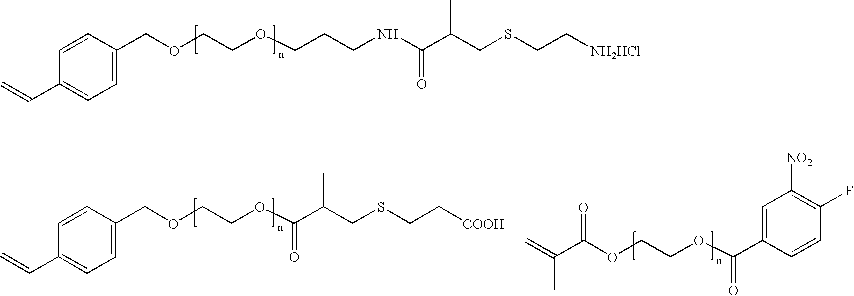

In another embodiment, the linking polymers do not incorporate a poly(ethylene glycol) backbone structure but contain the reactive functional group such as a thiol, a carboxylic acid, an amine, 4-fluoro-5-nitro-benzoate, or a carboxylic acid activated ester. Preferably, the reactive functional group is an amine, 4-fluoro-5-nitro-benzoate, or a carboxylic acid activated ester. The following is a list of preferred monomers to form linking polymers, but is not intended to an exhaustive and complete list of all linking polymers according to the present invention:

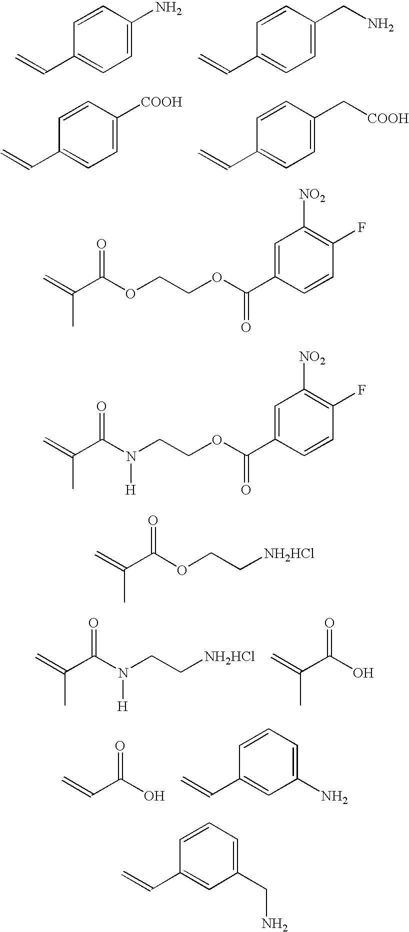

### Immobilized Dye

The dyes useful for imaging probes of the present invention are either attached to polymer shell of the nanoparticle or immobilized in the silica core. The dyes include both fluorescent dyes and quencher dyes. If immobilized in the silica core, the dye may contain functional groups that can react with the tetraorthosilicate and is immobilized in the silica core during its Stober synthesis. Specifically the functional group includes alkoxy silane or amino silane groups.

In one embodiment, the immobilized in the silica core dyes are fluorescent dyes and their quantum efficiency can be enhanced. Dyes such as cyanine dyes tend to form aggregates that do not fluoresce and fluorescence quantum yield decreases. By immobilizing the dye in the core of the silica core can reduce the aggregation and thus improve quantum efficiency. In such embodiment, the quencher dyes are attached via the cleavable spacer groups to the polymer shell. The fluorescence of the nanoparticle is quenched (quenched state) via fluorescence resonance energy transfer (FRET) between the donor fluorescent dye and acceptor quencher dye provided they are in close proximity. The imaging probe fluorescences (activated state) after the quencher dye is released from the polymer shell of the nanoparticle by enzyme specific cleavage.

In another embodiment, the quencher dye is immobilized in the silica core and the fluorescent dye is attached via the spacer groups on the polymer shell (quenched state) as shown in FIG. 2B. The fluorsescent dye fluorescences (activated state) once it is released from the polymer shell of the nanoparticle by cleavage.

In another embodiment, fluorescent dye is attached via the cleavable spacer groups to the polymer shell of the nanoparticle as shown in FIG. 2A. The imaging probe is in a quenched state because the fluorescent dye molecules are spatially near one another in close proximity. After some dye molecules are released from the nanoparticle by enzyme specific cleavage, the probe is activated and fluorescence detected.

Examples of suitable dyes include the following:

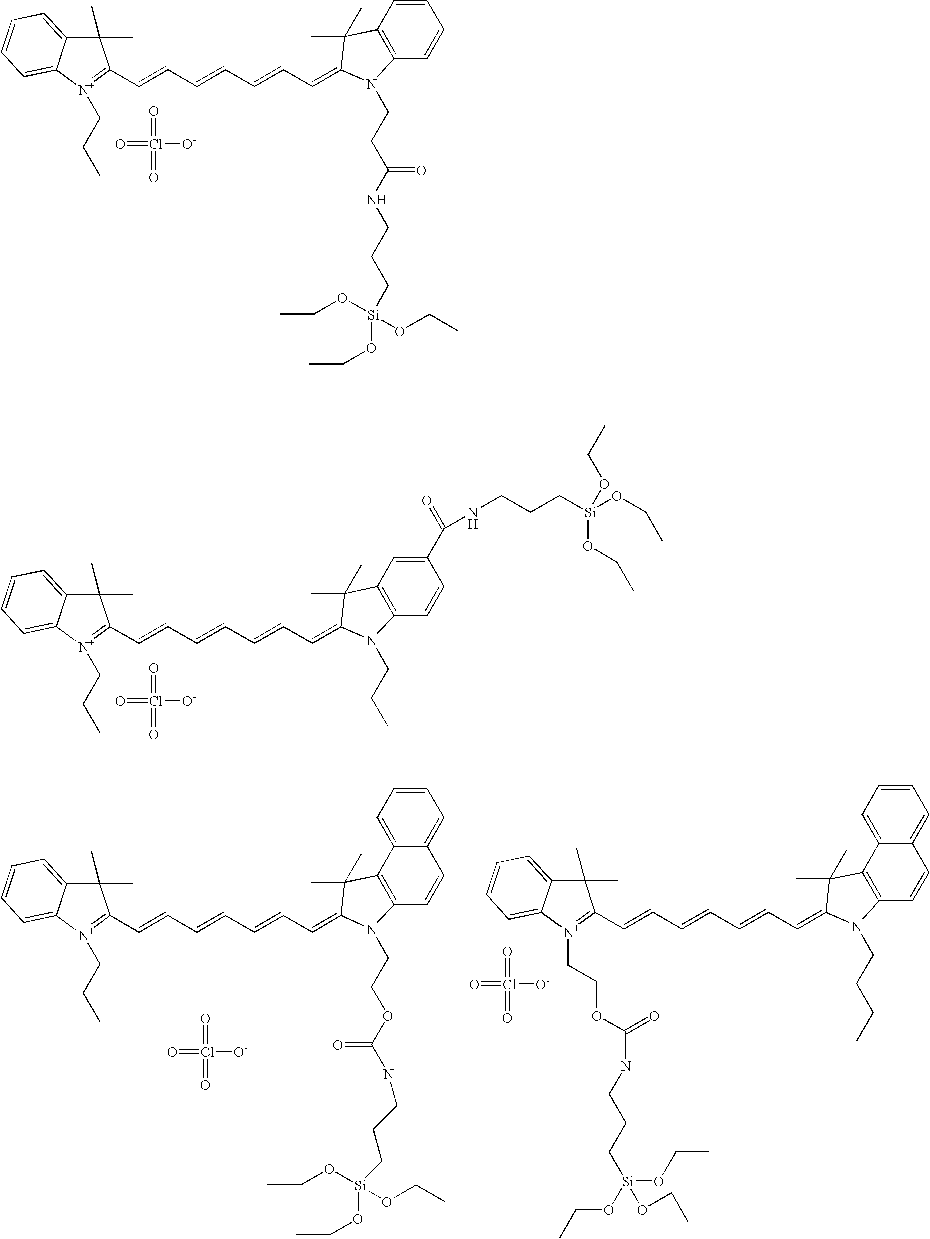

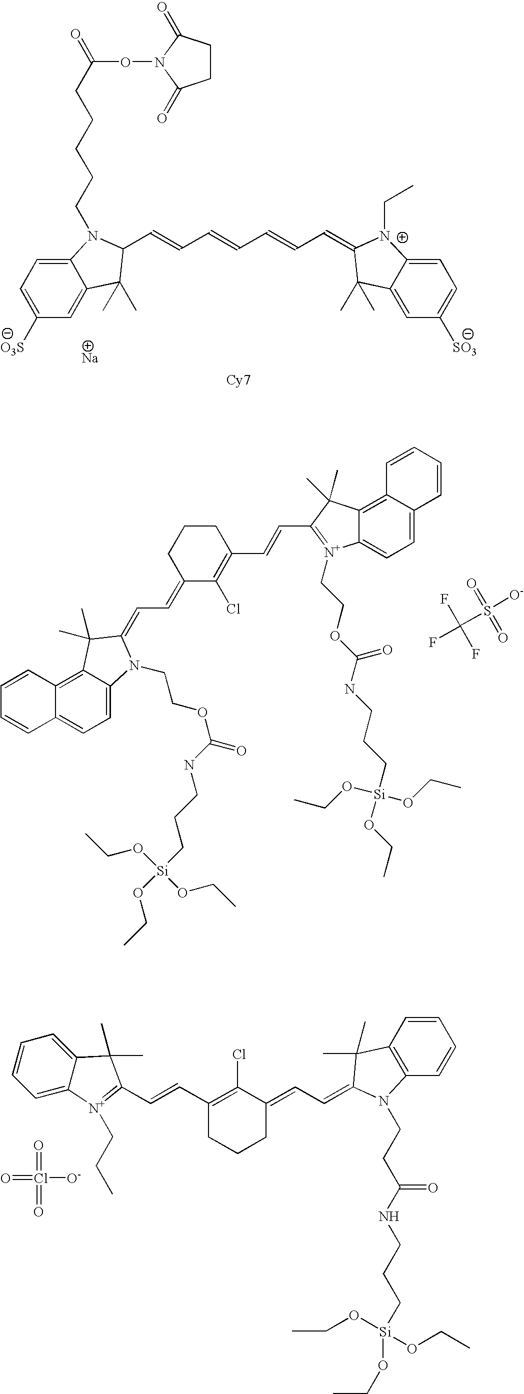

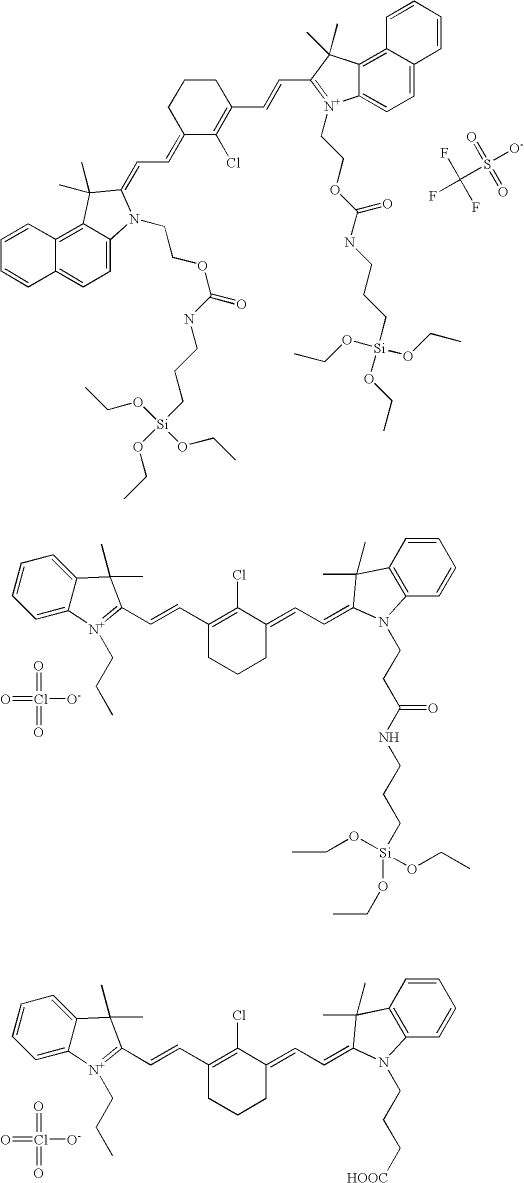

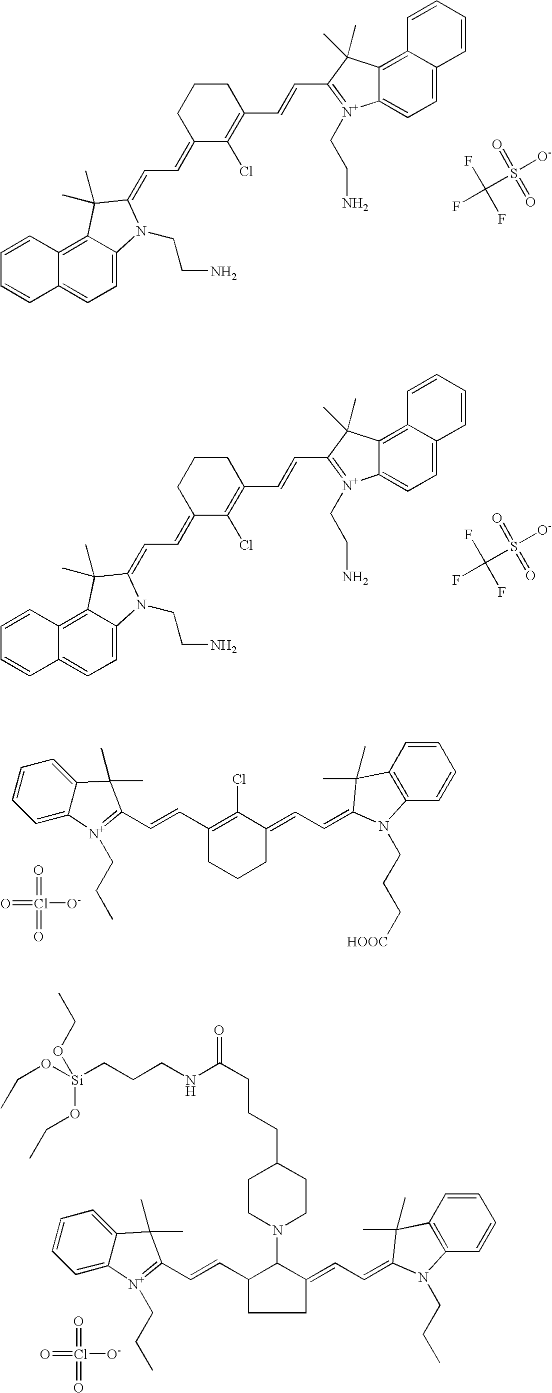

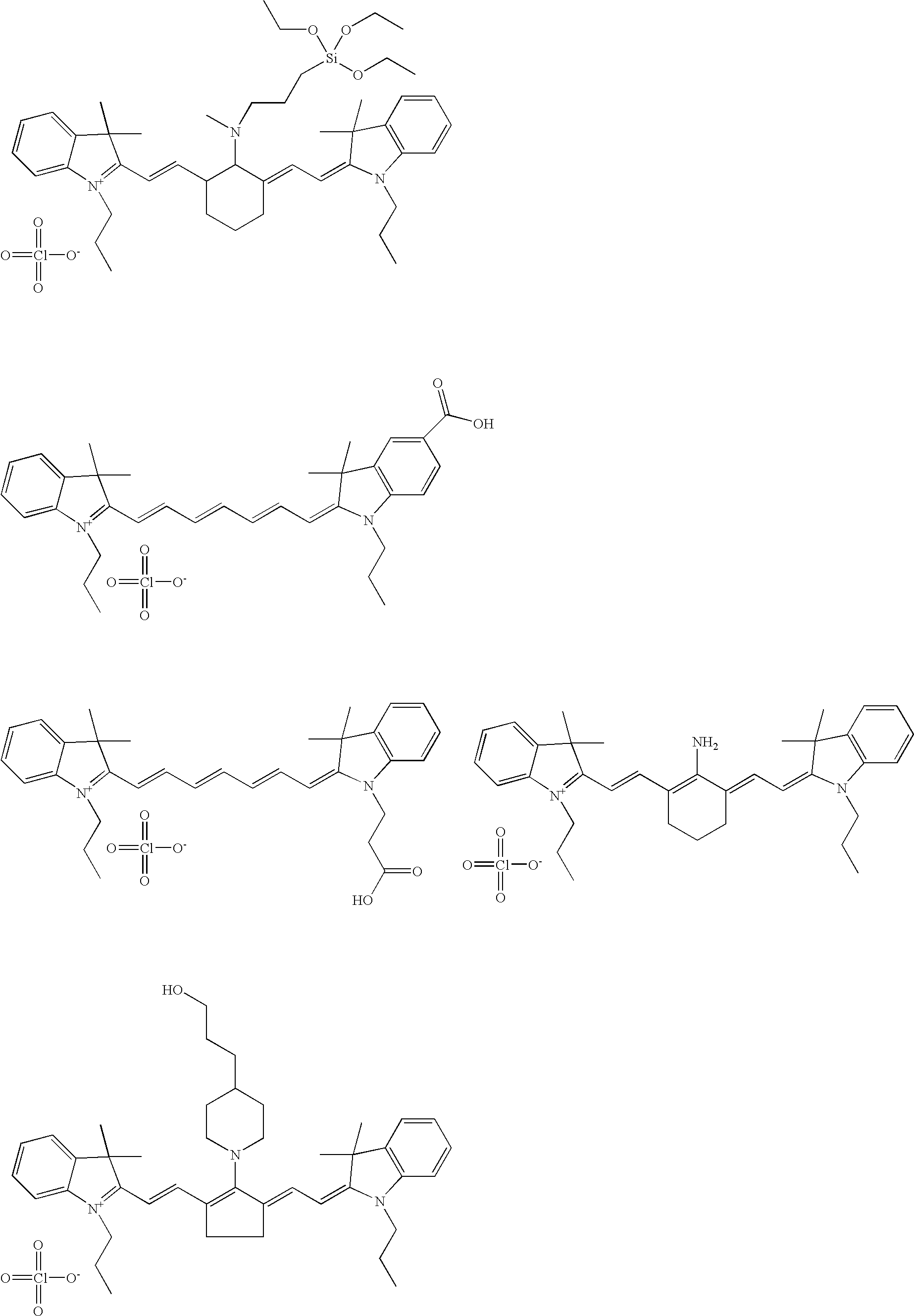

Dyes that are useful as fluorescent biomarkers or contrast agents emit significant fluorescent light during in vitro or in vivo diagnostic procedures. Many dyes do not emit fluorescent light because excitation energy is emitted as heat or non-fluorescent light. Of those dyes that do emit fluorescent energy, many are self quenched due to aggregation effects or have low quantum yields. Suitable fluorescent dyes that accumulate in diseased tissue (above all, in tumors) and that show a specific absorption and emission behavior may contribute towards enhancing the distinction of healthy from diseased tissue.

Examples of using dyes for in vivo diagnostics in humans are photometric methods of tracing in the blood to determine distribution areas, blood flow, or metabolic and excretory functions, and to visualize transparent structures of the eye (ophthalmology). Preferred dyes for such applications are indocyanine green and fluorescein (Googe, J. M. et al., Intraoperative Fluorescein Angiography; Ophthalmology, 100, (1993), 1167-70.

Indocyanine Green (Cardiogreen) is used for measuring the liver function, cardiac output and stroke volume, as well as the flood flow through organs and peripheral blood flows, (I. Med. 24 (1993), 10-27). In addition they are being tested as contrast media for tumor detection. Indocyanine green binds up to 100% to albumin and is mobilized in the liver. Fluorescent quantum efficiency is low in a hydrous environment. Its LD50 (0.84 mmol/kg) is high enough that strong anaphylactic responses may occur. Indocyanine green is unstable when dissolved and cannot be applied in saline media because precipitation will occur.

Photosensitizers designed for used in photodynamic therapy (PDT) (including haematopoporphyrin derivatives, photophrin II, benzopopphyrins, tetraphenyl porphyrins, chlorines, phthalocyanines) were used up to now for localizing and visualizing tumors (Bonnett R.; New photosensitizers for the photodymanic therapy of tumors, SPIE Vol. 2078 (1994)). It is a common disadvantage of the compounds listed that their absorption in the wavelentgth range of 650-1200 nm is only moderate. The phototoxicity required for PDT is disturbing for purely diagnostic purposes. Other patent specifications dealing with these topics are U.S. Pat. No. 4,945,239, WO 84/04665; WO 90/10219; DE-OS 4136769; and DE-PS 2910760.

Other dyes which have been developed for this purpose include: IRDye78, IrDye80, IRDye38, IRDye40, IRDye41, IRDye700, IRDye800, IRDye800CW, Cy5, Cy5.5, Cy7, IR-786, DRAQ5NO, Licor NIR, Alexa Fluor 680, Alexa Fluor 750, La Jolla Blue, quantum dots, as well as fluorphores described U.S. Pat. No. 6,083,875.

Typically, the dyes of the present invention are selected from the same family, such as the Oxonol, Pyryliuim, Squaric, Croconic, Rodizonic, polyazaindacenes or coumarins. Other suitable families of dyes include hydrocarbon and substituted hydrocarbon dyes; scintillation dyes (usually oxazoles and oxadiazoles); aryl- and heteroaryl-substituted polyolefins (C₂-C₈ olefin portion); merocyanines, carbocyanines; phthalocyanines; oxazines; carbostyryl; and porphyrin dyes. It is also possible, however, to achieve efficient energy transfer between different classes of dyes (dyes that are structurally different) such as between polyolefinic dyes and dipyrrometheneboron difluoride dyes, coumarin dyes and dipyrrometheneboron difluoride dyes, polyolefinic dyes and coumarin dyes; dipyrrometheneboron difluoride dyes and oxazine dyes; and many others.

Examples of commercially available dyes are listed below. Useful dyes of the present invention can be obtained from these dyes by further reaction to incorporate silane moieties for crosslinking. Useful parent dyes include 5-Amino-9-diethyliminobenzo(a)phenoxazonium Perchlorate; 7-Amino-4-methylcarbostyryl; 7-Amino-4-methylcoumarin; 7-Amino-4-trifluoromethylcoumarin; 3-(2′-Benzimidazolyl)-7-N,N-diethylaminocoumarin; 3-(2′-Benzothiazolyl)-7-diethylaminocoumarin; 2-(4-Biphenylyl)-5-(4-t-butylphenyl)-1,3,4-oxadiazole; 2-(4-Biphenylyl)-5-phenyl-1,3,4-oxadiazole; 2-(4-Biphenyl)-6-phenylbenzoxazole-1,3; 2,5-Bis-(4-biphenylyl)-1,3,4-oxadiazole; 2,5-Bis-(4-biphenylyl)-oxazole; 4,4′″-Bis-(2-butyloctyloxy)-p-quaterphenyl; p-Bis(o-methylstyryl)-benzene; 5,9-Diaminobenzo(a)phenoxazonium Perchlorate; 4-Dicyanomethylene-2-methyl-6-(p-dimethylarninostyryl)-4H-pyran; 1,1′-Diethyl-2,2′-carbocyanine Iodide; 1,1′-Diethyl-4,4′-carbocyanine Iodide; 3,3′-Diethyl-4,4′,5,5′-dibenzothiatricarbocyanine Iodide; 1,1′-Diethyl-4,4′-dicarbocyanine Iodide; 1,1′-Diethyl-2,2′-dicarbocyanine Iodide; 3,3′-Diethyl-9,11-neopentylenethiatricarbocyanine Iodide; 1,3′-Diethyl-4,2′-quinolyloxacarbocyanine Iodide; 1,3′-Diethyl-4,2′-quinolylthiacarbocyanine Iodide; 3-Diethylamino-7-diethyliminophenoxazonium Perchlorate; 7-Diethylamino-4-methylcoumarin; 7-Diethylamino-4-trifluoromethylcoumarin; 7-Diethylaminocoumarin; 3,3′-Diethyloxadicarbocyanine Iodide; 3,3′-Diethylthiacarbocyanine Iodide; 3,3′-Diethylthiadicarbocyanine Iodide; 3,3′-Diethylthiatricarbocyanine Iodide; 4,6-Dimethyl-7-ethylaminocoumarin; 2,2′″-Dimethyl-p-quaterphenyl; 2,2″-Dimethyl-p-terphenyl; 7-Dimethylamino-1-methyl-4-methoxy-8-azaquinolone-2; 7-Dimethylamino-4-methylquinolone-2; 7-Dimethylamino-4-trifluoromethylcoumarin; 2-(4-(4-Dimethylaminophenyl)-1,3-butadienyl)-3-ethylbenzothiazolium Perchlorate; 2-(6-(p-Dimethylaminophenyl)-2,4-neopentylene-1,3,5-hexatrienyl)-3-methylbenzothiazolium Perchlorate; 2-(4-(p-Dimethylaminophenyl)-1,3-butadienyl)-1,3,3-trimethyl-3H-indolium Perchlorate; 3,3′-Dimethyloxatricarbocyanine Iodide; 2,5-Diphenylfuran; 2,5-Diphenyloxazole; 4,4′-Diphenylstilbene; 1-Ethyl-4-(4-(p-Dimethylaminophenyl)-1,3-butadienyl)-pyridinium Perchlorate; 1-Ethyl-2-(4-(p-Dimethylaminophenyl)-1,3-butadienyl)-pyridinium Perchlorate; 1-Ethyl-4-(4-(p-Dimethylaminophenyl)-1,3-butadienyl)-quinolium Perchlorate; 3-Ethylamino-7-ethylimino-2,8-dimethylphenoxazin-5-ium Perchlorate; 9-Ethylamino-5-ethylamino-10-methyl-5H-benzo(a)phenoxazonium Perchlorate; 7-Ethylamino-6-methyl-4-trifluoromethylcoumarin; 7-Ethylamino-4-trifluoromethylcoumarin; 1,1′,3,3,3′,3′-Hexamethyl-4,4′,5,5′-dibenzo-2,2′-indotricarboccyanine Iodide; 1,1′,3,3,3′,3′-Hexamethylindodicarbocyanine Iodide; 1,1′,3,3,3′,3′-Hexamethylindotricarbocyanine Iodide; 2-Methyl-5-t-butyl-p-quaterphenyl; 3-(2′-N-Methylbenzimidazolyl)-7-N,N-diethylaminocoumarin; 2-(1-Naphthyl)-5-phenyloxazole; 2,2′-p-Phenylen-bis(5-phenyloxazole); 3,5,3′″″,5′″″-Tetra-t-butyl-p-sexiphenyl; 3,5,3″″,5″″-Tetra-t-butyl-p-quinquephenyl; 2,3,5,6-1H,4H-Tetrahydro-9-acetylquinolizino-<9,9a,1-gh>coumarin; 2,3,5,6-1H,4H-Tetrahydro-9-carboethoxyquinolizino-<9,9a,1-gh>coumarin; 2,3,5,6-1H,4H-Tetrahydro-8-methylquinolizino-<9,9a,1->coumarin; 2,3,5,6-1H,4H-Tetrahydro-9-(3-pyridyl)-quinolizino-<9,9a,1-gh>coumarin; 2,3,5,6-1H,4H-Tetrahydro-8-trifluoromethylquinolizino-<9,9a,1- gh>coumarin; 2,3,5,6-1H,4H-Tetrahydroquinolizino-<9,9a,1-gh>coumarin; 3,3′,2″,3′″-Tetramethyl-p-quaterphenyl; 2,5,2″″,5″″-Tetramethyl-p-quinquephenyl; P-terphenyl; P-quaterphenyl; Nile Red; Rhodamine 700; Oxazine 750; Rhodamine 800; IR 125; IR 144; IR 140; IR 132; IR 26; IR 5; Diphenylhexatriene; Diphenylbutadiene; Tetraphenylbutadiene; Naphthalene; Anthracene; Pyrene; Chrysene; Rubrene; Coronene; Phenanthrene; Fluorene; Aluminum phthalocyanine; Platinum octaethylporphyrin; and the like.

Other examples of fluorescent dyes ate listed below:

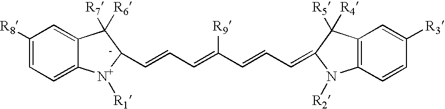

wherein R₁′, R₂′, R₃′, R₄′, R₅′, R₆′, R₇′, R₈′, and R₉′ are each independently selected from the group consisting of H, halogen, alkyl of from 1 to 20 carbon atoms, cycloalkyl of from 3 to 8 carbon atoms, heterocycloalkyl of from 2 to 8 carbon atoms, aryl or heteroaryl of from 4 to 20 carbon atoms, alkoxy, thioether, C(=Z)R, C(=Z)N(R)₂, COCl, amino, CN, nitro, oxiranyl, and glycidyl; wherein Z is O or NR, and R is an aryl or heteroary of from 4 to 20 carbon atoms, or an alkyl, alkynyl, or alkenyl of from 1 to 20 carbon atoms (preferably Z is O), and at least one of R₁′, R₂′, R₃′, R₄′, R₅′, R₆′, R₇′, R₈′, and R₉′ can be further reacted to form a silane moiety.

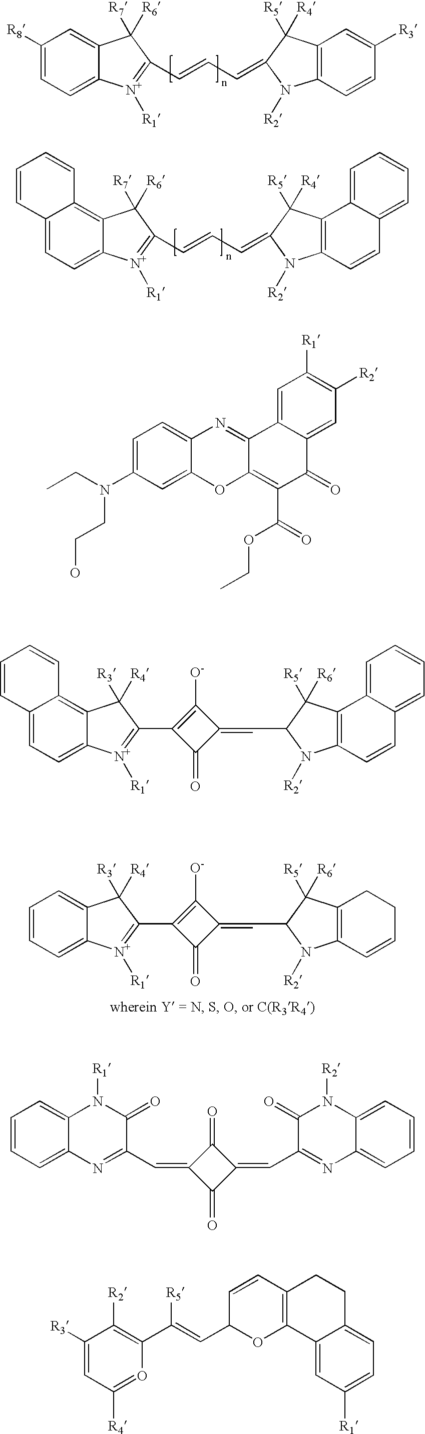

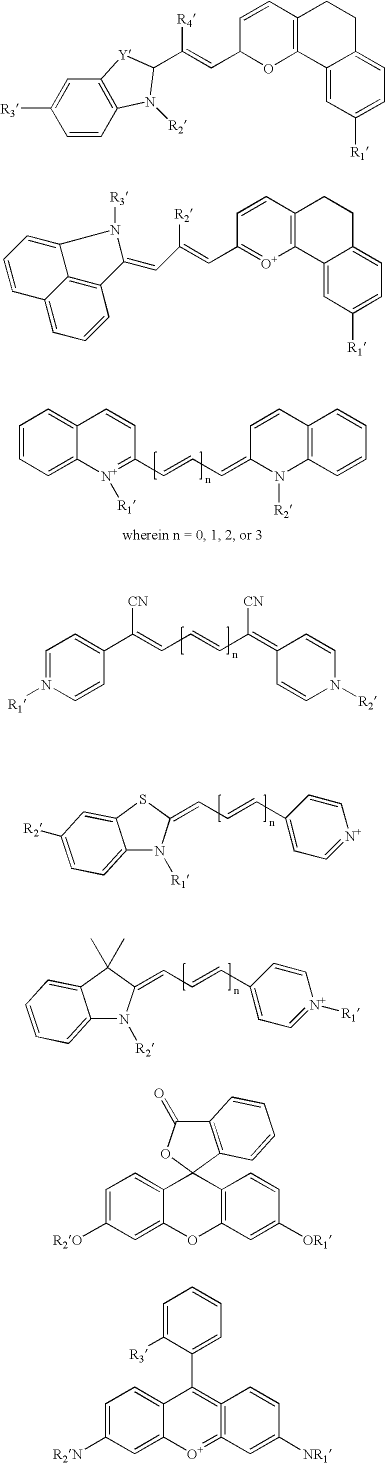

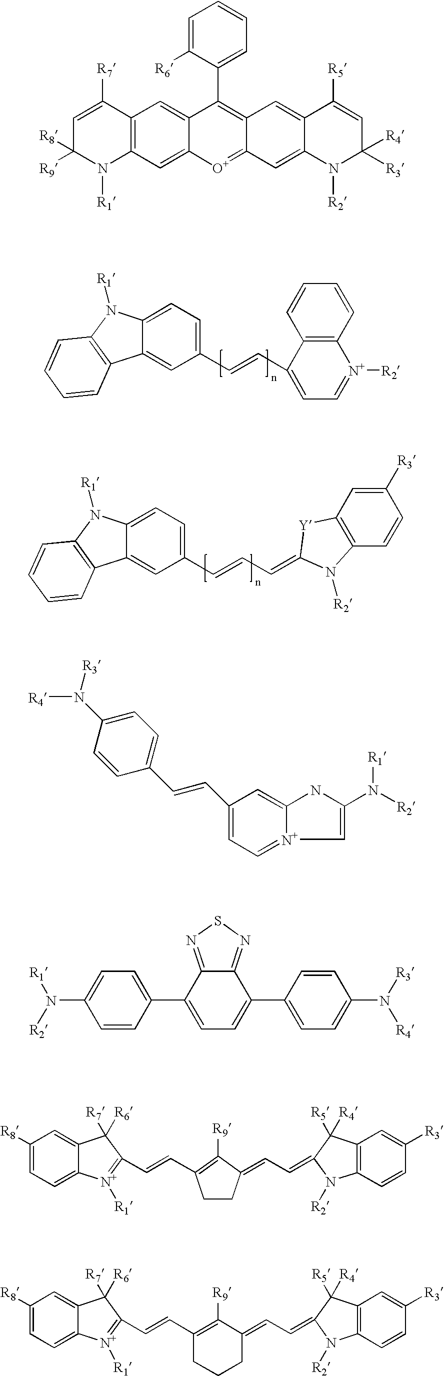

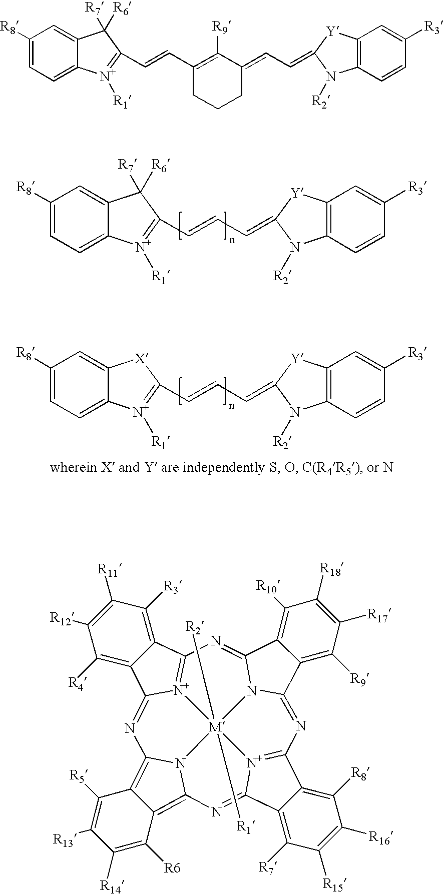

Wherein M′=Silicon, Magnesium, Aluminum, or Germanium; wherein R₁₀′, R₁₁′, R₁₂′, R₁₃′, R₁₄′, R₁₅′, R₁₆′, R₁₇′, and R₁₈′ are defined as R₁′, R₂′, R₃′, R₄′, R₅′, R₆′, R₇′, R₈′, and R₉′ above.

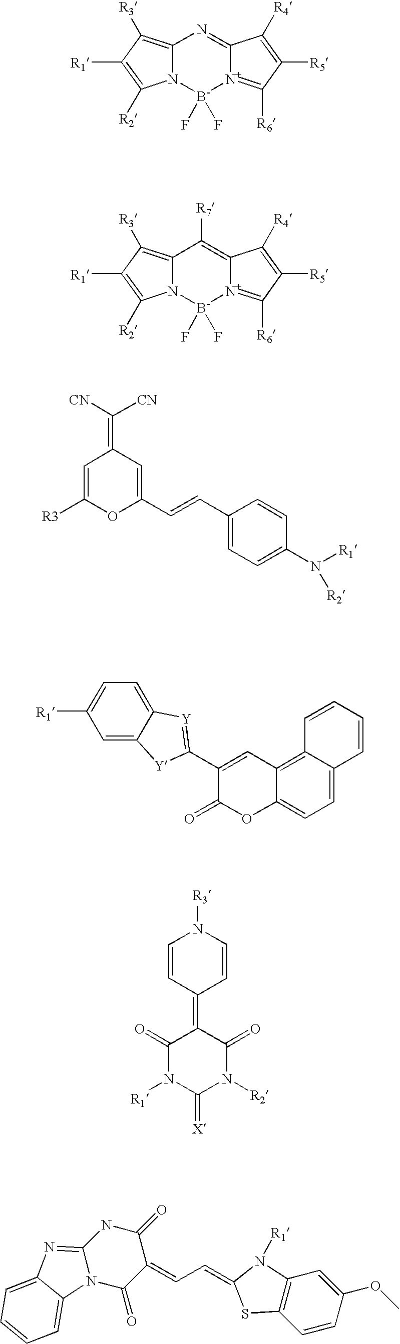

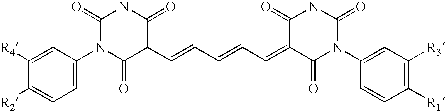

Many dyes do not emit fluorescent light because excitation energy is emitted as heat or non-fluorescent light. Of those dyes that do emit fluorescent energy, many are self quenched due to aggregation effects or have low quantum yields. These dyes are usually used as quencher dyes. Specifically, the quencher dye is represented by the following formulae:

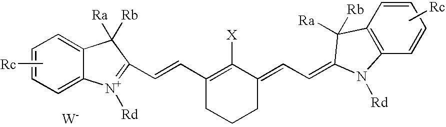

Wherein X is Cl, or aryl-substituted S, O, or N; Ra and Rb are substituted or unsubstituted alkyl and may form a ring; Rc is hydrogen or SO₃⁻, aryl, alkyl, alkoxy, or halogen and may form a fused ring with indole; and Rd is substituted or unsubstituted alkyl. At least one of the substituents is a linking group selected from a list of OH, COOH, NH₂, Si(OEt)₃, N₃, terminal alkyne, maleimide, thiol, isocyanate, isothiocyanate.

The following are some specific examples for such quencher molecules:

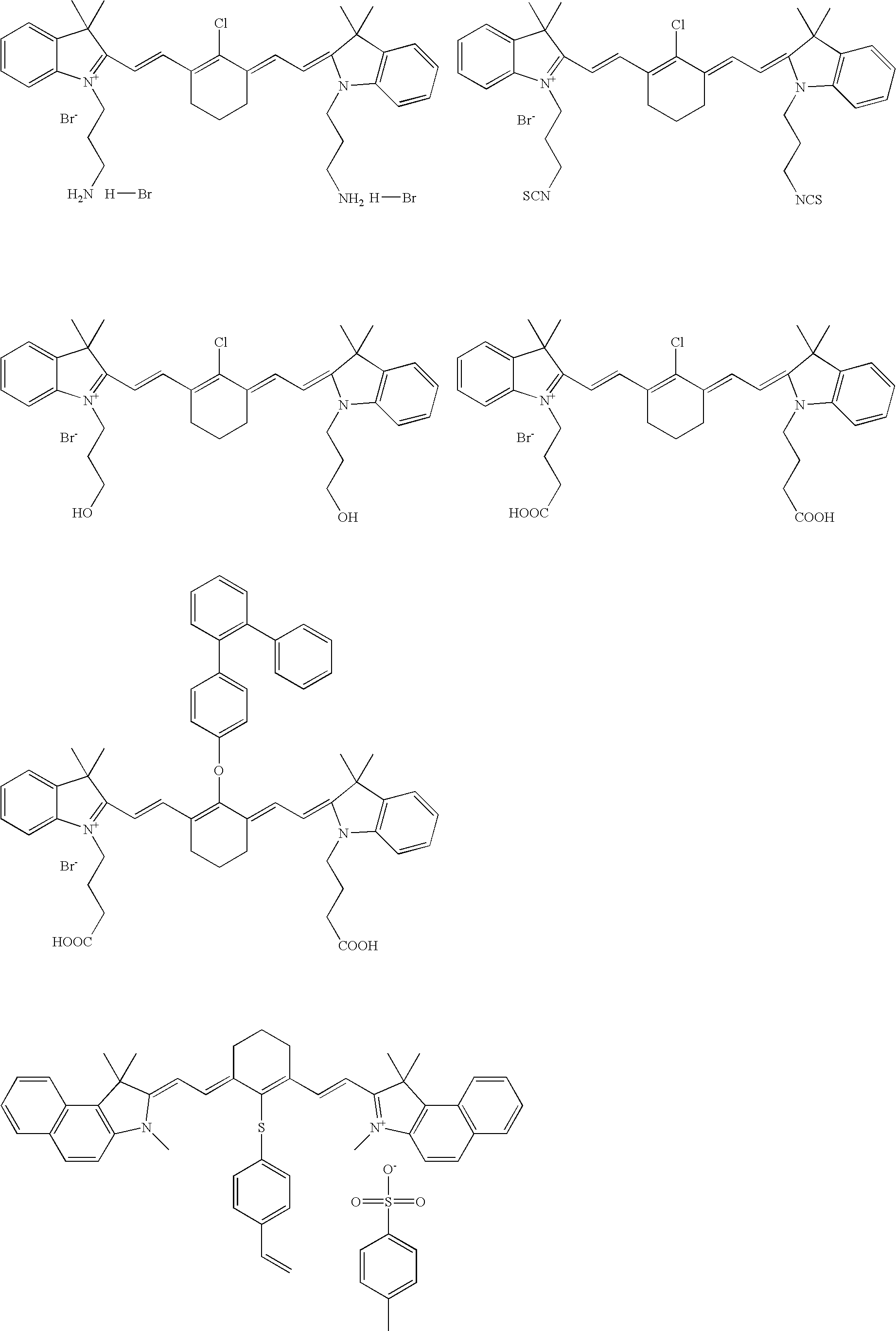

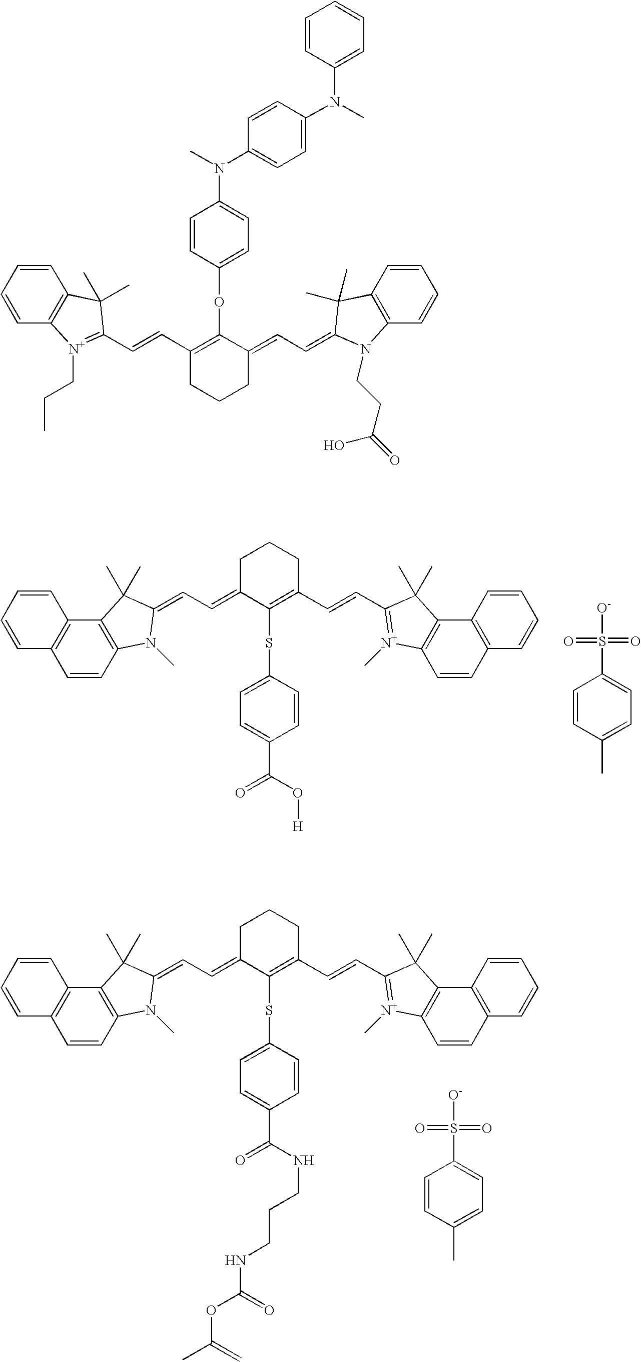

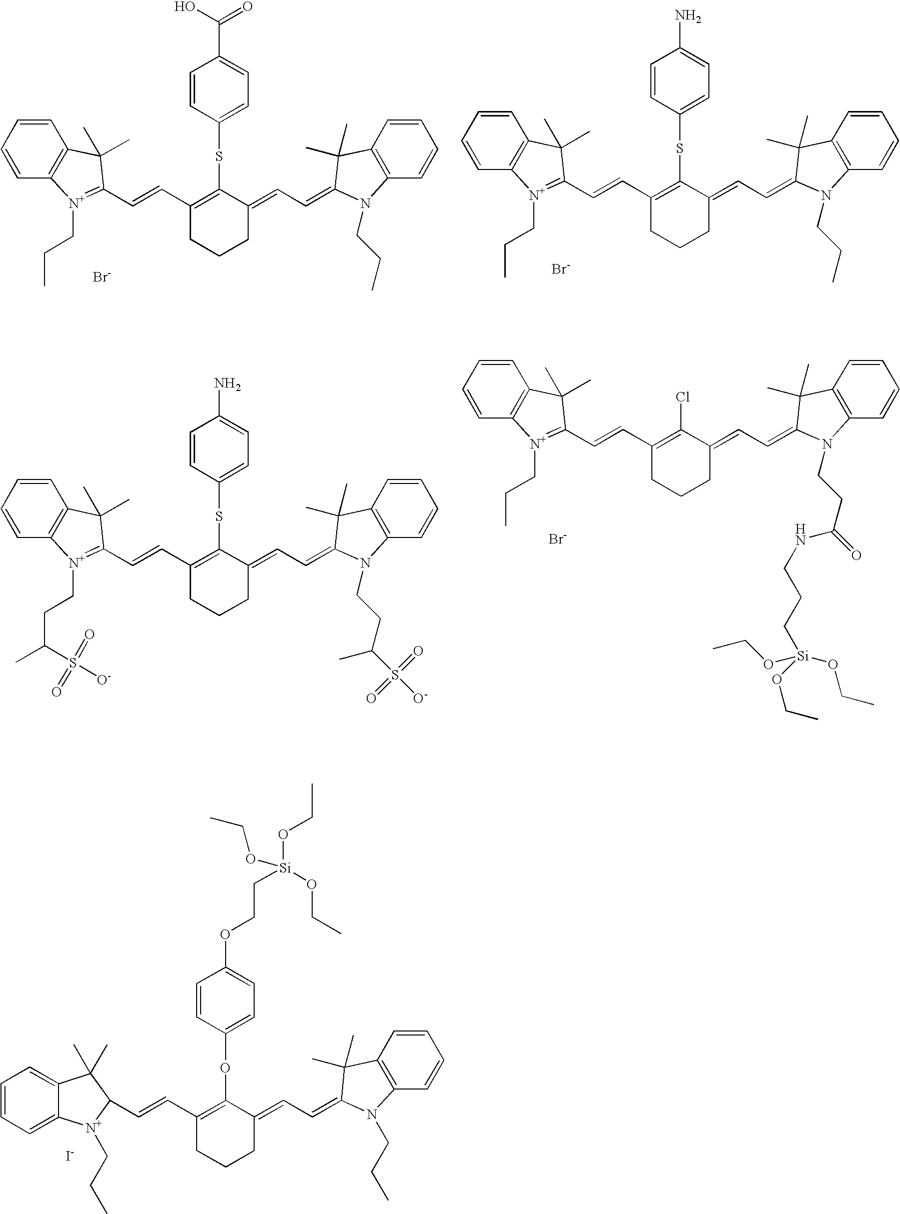

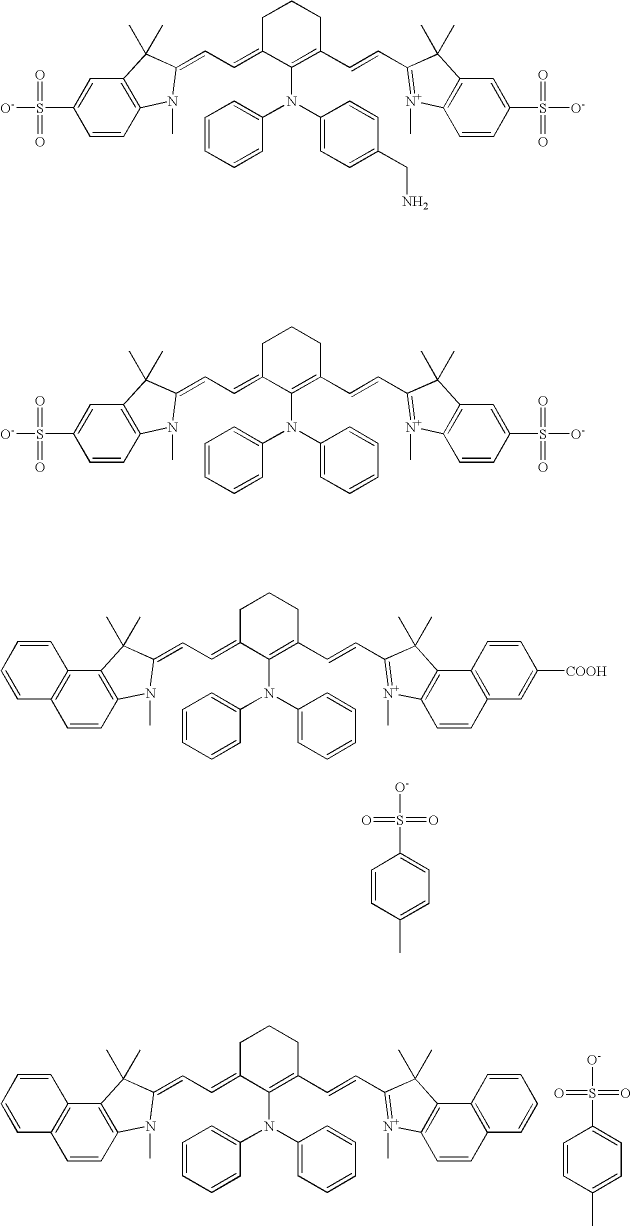

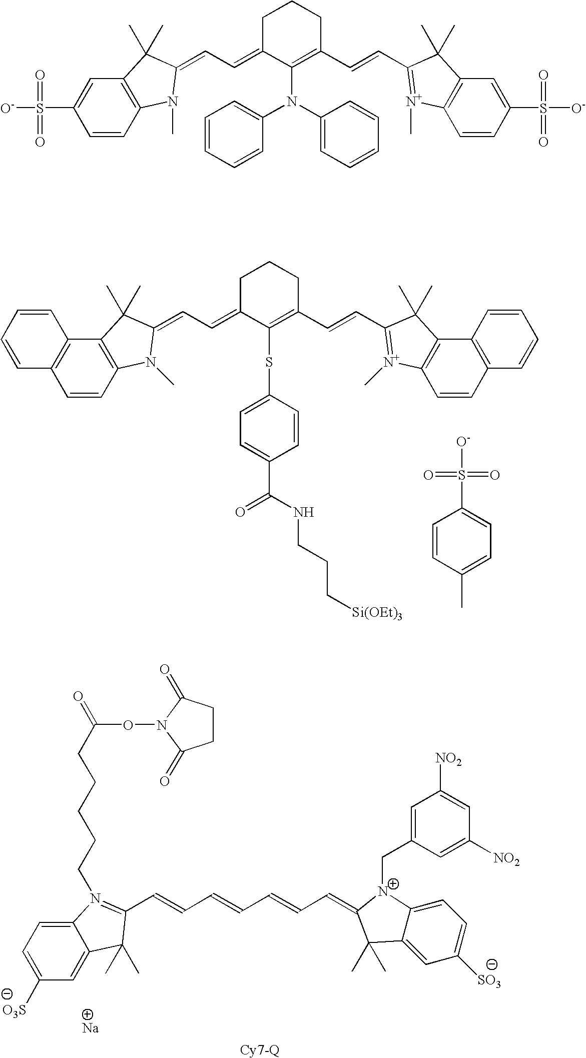

The imaging probes of the present invention are optically silent (quenched, no fluorescence) in their native (quenched) state and become highly fluorescent after enzyme-mediated release of dyes. The dye of the imaging probe of the present invention can be attached via an enzyme-specific cleavable spacer to the polymer shell. Enzyme specificity is imparted through the use of enzyme cleavage-specific peptide sequences, which can be varied depending upon the desired protease to be visualized. Moreover, other enzymatic pathways are amenable to this activation scheme. This approach has several major advantages over simple targeting: (1) a single enzyme molecule can cleave multiple dyes, resulting in signal amplification; (2) reduction of background signal of several orders of magnitude is possible because the quenched probe is optically silent until it is activated by its target; and (3) very specific enzyme activities can be potentially interrogated. All of these lead to better imaging visualization of tumors based on their enzyme over-expression profile because in most cancer and disease cells, the levels of certain proteases are highly elevated.

Many tumors have been shown to have elevated levels of proteolytic enzymes (protease) in adaptation to rapid cell cycling and for secretion to sustain invasion, metastasis formation, and angiogenesis. Because they are present at high levels in tumors and are elevated at an early stage, their type and level are tightly associated with specific cells or physiological or pathological process, proteases represent an attractive target for anti-tumor imaging and therapeutic strategies. Also they are much richer than DNA and mRNA in their concentrations.

MMPs are one of the over-expressed proteases in cancers. They are one of the most attractive diagnostic markers, since their overexpressions are tightly associated with the aggressive growth of the cancer cells. Thus, their detection can serve as a surrogate marker for tumor staging, metastasis and recurrence. They can also be used to examine the effectiveness of therapeutic inhibitors. Specifically MMP-2 and MMP-9 are attractive imaging targets due to their critical roles in angiogenesis and metastasis. Elevated levels of MMP-2 and MMP-9 have been correlated with increased aggressiveness of tumor cells. MMP-2 has been observed to be overexpressed in more aggressive tumor cell.

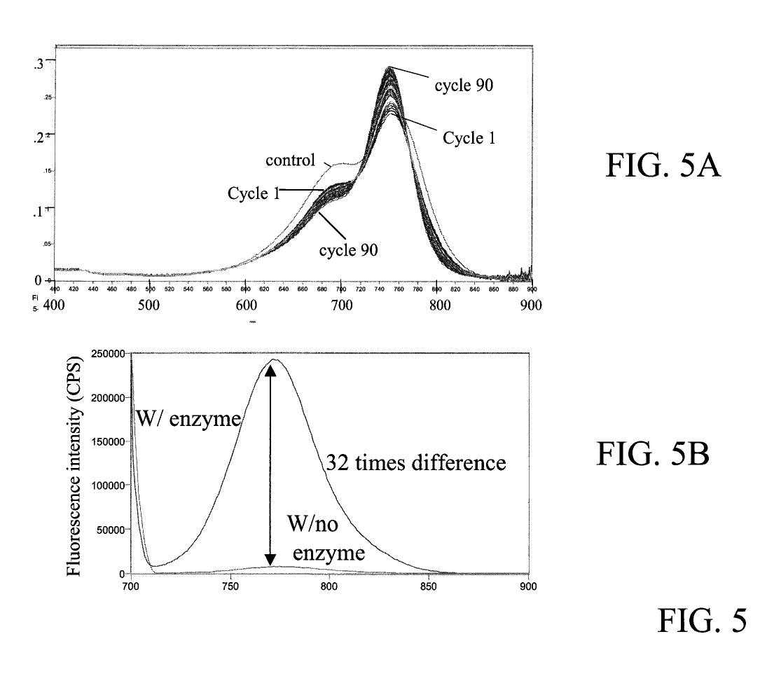

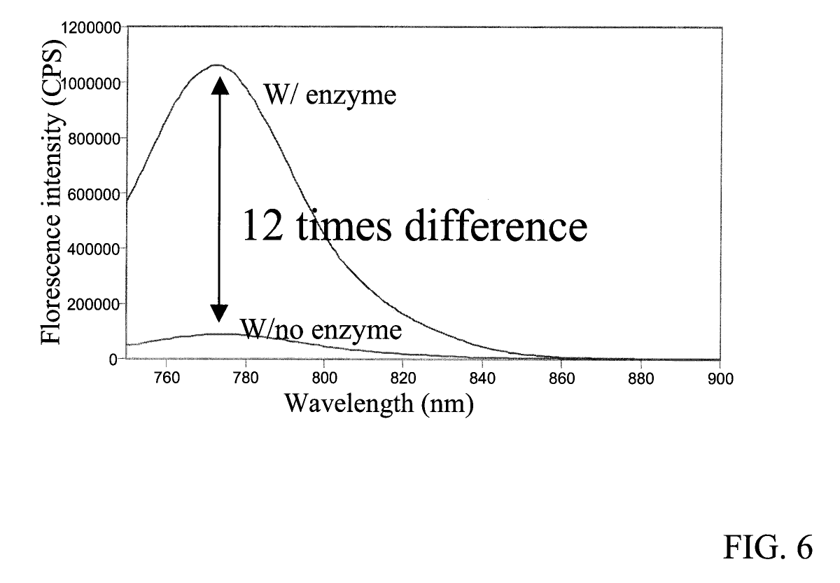

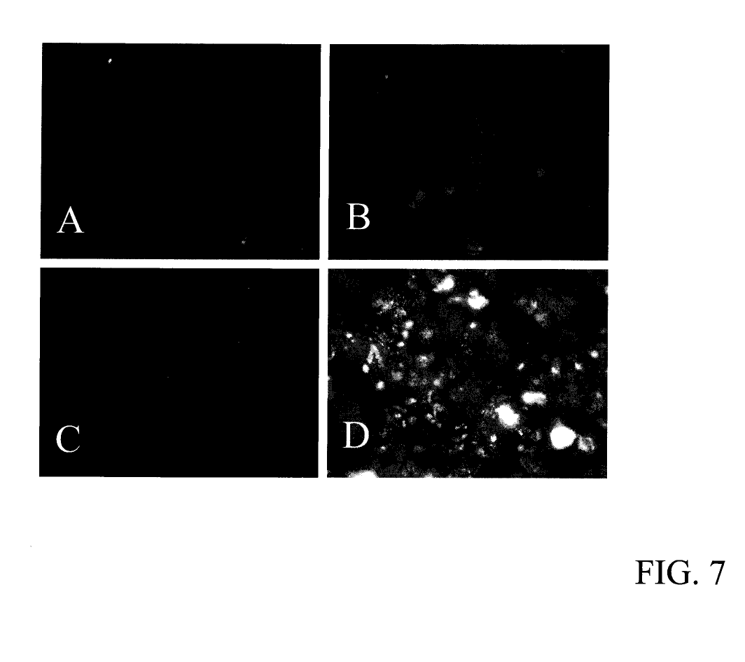

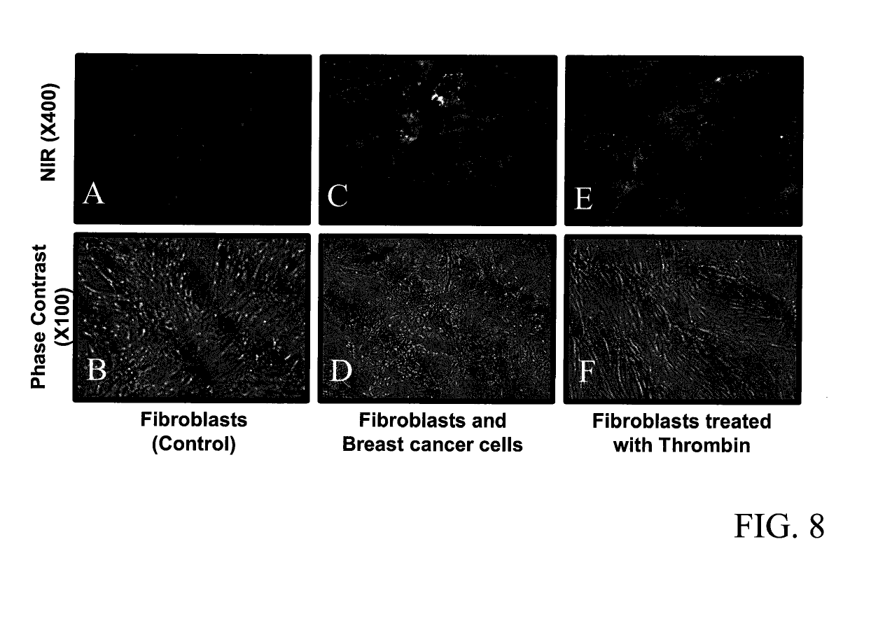

Thus, spacer groups containing peptide sequences recognized by MMP-2 can be used to produce a near infrared probe that undergoes fluorescence activation specifically in tumor tissues. The effectiveness of the activation by MMP-2 was examined by using breast cancer MCF-7 as model cancer cells, and thrombin to induce the activation of MMP-2 expressed by fibroblast cells as shown in FIG. 7 and FIG. 8. Peptide sequence used as spacer groups and recognized by MMP-2 of the present invention include oligopeptides such that those disclosed in International Publication No. WO2004/026344. FIG. 5 and FIG. 6 shows the activation of the imaging probe by incorporating MMP-2-specific peptide sequence via self-quenching and FRET mechanism. FIG. 5, which includes FIGS. 5A and 5B, shows a mechanistic study of enzymatic activation of imaging probe by self-quenching. FIG. 5A illustrates a time-series record of absorbance of peptide-dye conjugates loaded nanoparticle imaging probe incubated with enzyme (240 μl solution with 0.2 μg enzyme MMP-2, 1 mm cell), which shows a gradual decrease of dimeric peak and increase of monomeric absorbance peak. FIG. 5B depicts the fluorescence of peptide-dye conjugates loaded nanoparticles before and after incubation with enzyme MMP-2, which show 32 times of fluorescence increase. FIG. 6 illustrates FRET based fluorescence gain after incubation of activatable imaging probe incubation with enzyme MMP-2, which show 12 times of fluorescence increase.

FIG. 7 depicts the detection of Matrix Metalloproteinase-2 (MMP-2) activity in Breast cancer MCF-7 cells A) and B) are fluorescence Image; C) and D) are NIR imaging. A) and C) are images of activatable imaging probe with Cy7 attached silica nanoparticle (no peptide spacer); B) and D) are images of peptide-dye conjugate loaded nanoparticle.

FIG. 8 depicts NIR (A, C, E) and phase contrast imaging (B, D, F) of fibroblasts cells in the presence of activatable nanoprobes. A), B) Control, no activation component added; C), D) Breast cancer MCF-7 cells added, induced MMP-2 activation in fibroblasts; E), F) Thrombin added, which induced MMP-2 activation in fibroblasts.

Various other enzymes can be exploited to provide probe activation (cleavage of spacer groups to release dye) in particular target tissues in particular diseases as disclosed in the prior art and in a publication by Mahmood et al. (Mahmood, U. and Weissleder, R. Molecular Cancer Therapeutics 2003, 2, 489-496).

Other diagnostic agents (beside the dyes disclosed above), such as therapeutic or targeting agents, can also be attached to the imaging probe of the present invention via enzyme-specific spacer groups and be released from the imaging probe for imaging and therapeutic application

The present nanoparticles can also be useful as a carrier for carrying a biological, pharmaceutical or diagnostic component. Specifically, the nanoparticle used as a carrier does not necessarily encapsulate a specific therapeutic or an imaging component, but rather serves as a carrier for the biological, pharmaceutical or diagnostic components. Biological, pharmaceutical or diagnostic components such as therapeutic agents, diagnostic agents, dyes or radiographic contrast agents. The term “diagnostic agent” includes components that can act as contrast agents and thereby produce a detectable indicating signal in the host mammal. The detectable indicating signal may be gamma-emitting, radioactive, echogenic, fluoroscopic or physiological signals, or the like. The term biomedical agent, as used herein, includes biologically active substances which are effective in the treatment of a physiological disorder, pharmaceuticals, enzymes, hormones, steroids, recombinant products, and the like. Exemplary therapeutic agents are antibiotics, thrombolytic enzymes such as urokinase or streptokinase, insulin, growth hormone, chemotherapeutics such as adriamycin and antiviral agents such as interferon and acyclovir. Upon enzymatic degradation, such as by a protease or a hydrolase, the therapeutic agents can be released over a period of time. A variety of drugs with diverse characteristics, including genes and proteins, can also be incorporated into the imaging probe of the present invention and released upon activation.

The distribution of drug-loaded imaging probe in the body may be determined mainly by their size and surface properties and these are less affected by the properties of loaded drugs if they are embedded in the inner core of the nanoparticle. In this regard, the design of the size and surface properties of polymer shell of the imaging probe have crucial importance in achieving modulated drug delivery with remarkable efficacy. Functionalization of the polymer shell of the imaging probe to modify its physicochemical and biological properties is of great value from the standpoint of designing the carrier systems for receptor-mediated drug and gene delivery. Included within the scope of the invention are compositions comprising the core-shell nanoparticle of the current invention and a suitable targeting molecule. As used herein, the term “targeting molecule” refers to any molecule, atom, or ion linked to the polymer shell of the nanoparticle of the current invention that enhances binding, transport, accumulation, residence time, bioavailability or modifies biological activity of the polymer networks or biologically active compositions of the current invention in the body or cell. The targeting molecule will frequently comprise an antibody, fragment of antibody or chimeric antibody molecules typically with specificity for a certain cell surface antigen. It could also be, for instance, a hormone having a specific interaction with a cell surface receptor, or a drug having a cell surface receptor. For example, glycolipids could serve to target a polysaccharide receptor. It could also be, for instance, enzymes, lectins, and polysaccharides. Low molecular mass ligands, such as folic acid and derivatives thereof are also useful in the context of the current invention. The targeting molecules can also be polynucleotide, polypeptide, peptidomimetic, carbohydrates including polysaccharides, derivatives thereof or other chemical entities obtained by means of combinatorial chemistry and biology. Targeting molecules can be used to facilitate intracellular transport of the nanoparticles of the invention, for instance transport to the nucleus, by using, for example, fusogenic peptides as targeting molecules described by Soukchareun et al., Bioconjugate Chem., 6, 43, (1995) or Arar et al., Bioconjugate Chem., 6, 43 (1995), caryotypic peptides, or other biospecific groups providing site-directed transport into a cell (in particular, exit from endosomic compartments into cytoplasm, or delivery to the nucleus).

The described composition can further comprise a biological, pharmaceutical or diagnostic component that includes a targeting moiety that recognizes the specific target cell. Recognition and binding of a cell surface receptor through a targeting moiety associated with a described nanoparticle used as a carrier can be a feature of the described compositions. For purposes of the present invention, a compound carried by the nanoparticle may be referred to as a “carried” compound. For example, the biological, pharmaceutical or diagnostic component that includes a targeting moiety that recognizes the specific target cell described above is a “carried” compound. This feature takes advantage of the understanding that a cell surface binding event is often the initiating step in a cellular cascade leading to a range of events, notably receptor-mediated endocytosis. The term “receptor mediated endocytosis” (“RME”) generally describes a mechanism by which, catalyzed by the binding of a ligand to a receptor disposed on the surface of a cell, a receptor-bound ligand is internalized within a cell. Many proteins and other structures enter cells via receptor mediated endocytosis, including insulin, epidermal growth factor, growth hormone, thyroid stimulating hormone, nerve growth factor, calcitonin, glucagon and many others.

Receptor Mediated Endocytosis affords a convenient mechanism for transporting a described nanoparticle, possibly containing other biological, pharmaceutical or diagnostic components, to the interior of a cell. In receptor mediated endocytosis (RME), the binding of a ligand by a receptor disposed on the surface of a cell can initiate an intracellular signal, which can include an endocytosis response. Thus, a nanoparticle used as a carrier with an associated targeting moiety, can bind on the surface of a cell and subsequently be invaginated and internalized within the cell. A representative, but non-limiting, list of moieties that can be employed as targeting agents useful with the present compositions includes proteins, peptides, aptomers, small organic molecules, toxins, diptheria toxin, pseudomonas toxin, cholera toxin, ricin, concanavalin A, Rous sarcoma virus, Semliki forest virus, vesicular stomatitis virus, adenovirus, transferrin, low density lipoprotein, transcobalamin, yolk proteins, epidermal growth factor, growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysacchorides. Homologs or fragments of the presented moieties can also be employed. These targeting moieties can be associated with a nanoparticle and be used to direct the nanoparticle to a target cell, where it can subsequently be internalized. There is no requirement that the entire moiety be used as a targeting moiety. Smaller fragments of these moieties known to interact with a specific receptor or other structure can also be used as a targeting moiety.

An antibody or an antibody fragment represents a class of most universally used targeting moiety that can be utilized to enhance the uptake of nanoparticles into a cell. Antibodies may be prepared by any of a variety of techniques known to those of ordinary skill in the art. Antibodies can be produced by cell culture techniques, including the generation of monoclonal antibodies or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, (Eur. J. Immunol. 1976, 6511-519), and improvements thereto.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described (Winter et al. Nature 1991, 349,293-299; Lobuglio et al. Proc. Nat. Acad. Sci. USA 1989, 86, 4220-4224). These “humanized” molecules are designed to minimize unwanted immunological response toward rodent antihuman antibody molecules that limits the duration and effectiveness of therapeutic applications of those moieties in human recipients.

Vitamins and other essential minerals and nutrients can be utilized as targeting moiety to enhance the uptake of nanoparticles by a cell. In particular, a vitamin ligand can be selected from the group consisting of folate, folate receptor-binding analogs of folate, and other folate receptor-binding ligands, biotin, biotin receptor-binding analogs of biotin and other biotin receptor-binding ligands, riboflavin, riboflavin receptor-binding analogs of riboflavin and other riboflavin receptor-binding ligands, and thiamin, thiamin receptor-binding analogs of thiamin and other thiamin receptor-binding ligands. Additional nutrients believed to trigger receptor mediated endocytosis, and thus also having application in accordance with the presently disclosed method, are carnitine, inositol, lipoic acid, niacin, pantothenic acid, pyridoxal, and ascorbic acid, and the lipid soluble vitamins A, D, E and K. Furthermore, any of the “immunoliposomes” (liposomes having an antibody linked to the surface of the liposome) described in the prior art are suitable for use with the described compositions.

Since not all natural cell membranes possess biologically active biotin or folate receptors, use of the described compositions in vitro on a particular cell line can involve altering or otherwise modifying that cell line first to ensure the presence of biologically active biotin or folate receptors. Thus, the number of biotin or folate receptors on a cell membrane can be increased by growing a cell line on biotin or folate deficient substrates to promote biotin and folate receptor production, or by expression of an inserted foreign gene for the protein or apoprotein corresponding to the biotin or folate receptor.

Receptor mediated endocytosis (RME) is not the exclusive method by which the described nanoparticle can be translocated into a cell. Other methods of uptake that can be exploited by attaching the appropriate entity to a nanoparticle include the advantageous use of membrane pores. Phagocytotic and pinocytotic mechanisms also offer advantageous mechanisms by which a nanoparticle can be internalized inside a cell.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

A cell surface recognition sequence is not a requirement. Thus, although a cell surface receptor targeting moiety can be useful for targeting a given cell type, or for inducing the association of a described nanoparticle with a cell surface, there is no requirement that a cell surface receptor targeting moiety be present on the surface of a nanoparticle.

After a sufficiently pure nanoparticle, preferably comprising a nanoparticle with a biological, pharmaceutical or diagnostic component, has been prepared, it might be desirable to prepare the nanoparticle in a pharmaceutical composition that can be administered to a subject or sample. Preferred administration techniques include parenteral administration, intravenous administration and infusion directly into any desired target tissue, including but not limited to a solid tumor or other neoplastic tissue. Purification can be achieved by employing a final purification step, which dissolves the nanoparticle in a medium comprising a suitable pharmaceutical composition. Suitable pharmaceutical compositions generally comprise an amount of the desired nanoparticle with active agent in accordance with the dosage information (which is determined on a case-by-case basis). The described nanoparticles are admixed with an acceptable pharmaceutical diluent or excipient, such as a sterile aqueous solution, to give an appropriate final concentration. Such formulations can typically include buffers such as phosphate buffered saline (PBS), or additional additives such as pharmaceutical excipients, stabilizing agents such as BSA or HSA, or salts such as sodium chloride.

For parenteral administration it is generally desirable to further render such compositions pharmaceutically acceptable by insuring their sterility, non-immunogenicity and non-pyrogenicity. Such techniques are generally well known in the art. Moreover, for human administration, preparations should meet sterility, pyrogenicity, general safety and purity standards as required by FDA Office of Biological Standards. When the described nanoparticle composition is being introduced into cells suspended in a cell culture, it is sufficient to incubate the cells together with the nanoparticle in an appropriate growth media, for example Luria broth (LB) or a suitable cell culture medium. Although other introduction methods are possible, these introduction treatments are preferable and can be performed without regard for the entities present on the surface of a nanoparticle used as a carrier.

Included within the scope of the invention are compositions comprising nanoparticles of the current invention and other suitable imagable moieties. The nature of the imagable moiety depends on the imaging modality utilized in the diagnosis. The imagable moiety must be capable of detection either directly or indirectly in an in vivo diagnostic imaging procedure, for example, moieties which emit or may be caused to emit detectable radiation (e.g. by radioactive decay, fluorescence excitation, spin resonance excitation, etc.), moieties which affect local electromagnetic fields (e.g. paramagnetic, superparamagnetic, ferrimagnetic or ferromagnetic species), moieties which absorb or scatter radiation energy (e.g. chromophores, particles (including gas or liquid containing vesicles), heavy elements and compounds thereof, etc.), and moieties which generate a detectable substance (e.g. gas microbubble generators), etc.

A very wide range of materials detectable by diagnostic imaging modalities is known from the art. Thus, for example, for ultrasound imaging an echogenic material, or a material capable of generating an echogenic material will normally be selected, for X-ray imaging the imagable moieties will generally be or contain a heavy atom (e.g. of atomic weight 38 or above), for magnetic resonance imaging (MRI) the imagable moieties will either be a non zero nuclear spin isotope (such as ¹⁹F) or a material having unpaired electron spins and hence paramagnetic, superparamagnetic, ferrimagnetic or ferromagnetic properties, for light imaging the imagable moieties will be a light scatterer (e.g. a colored or uncolored particle), a light absorber or a light emitter, for magnetometric imaging the imagable moieties will have detectable magnetic properties, for electrical impedance imaging the imagable moieties will affect electrical impedance and for scintigraphy, SPECT, PET etc. the imagable moieties will be a radionuclide.

Examples of the suitable imagable moieties are widely known from the diagnostic imaging literature, e.g. magnetic iron oxide particles, gas-containing vesicles, chelated paramagnetic metals (such as Gd, Dy, Mn, Fe etc.). Particularly preferred imagable moieties are: chelated paramagnetic metal ions such as Gd, Dy, Fe, and Mn, especially when chelated by macrocyclic chelant groups (e.g. tetraazacyclododecane chelants such as 1,4,7,10-tetraazacyclododecane-N,N′,N″,N′″-tetraacetic acid (DOTA), 1,4,7,10-tetraazacyclododecane-N,N′,N″-triacetic acid (D03A), HP-D03A (10-(2-hydroxypropyl)-1,4,7,10-tetraazacyclododecane-1,4,7 triacetic acid) and analogues thereof; or by linker chelant groups such as DTPA (N,N,N′,N″,N″-diethylene-triaminepentaacetic acid (DTPA), DTPA-BMA (N,N,N′,N″,N″-diethylenetriaminepentaacetic acid bismethylamide), DPDP (N,N′-dipyridoxylethylenediamine-N,N′-diacetate-5,5′- bis(phosphate), ethylenediamine-N,N,N′,N′-tetraacetic acid (EDTA), 1-oxa-4,7,10-triazacyclododecane-N,N′,N″-triacetic acid (OTTA), trans(1,2)-cyclohexanodiethylene-triamine-pentaacetic acid (CDTPA), etc; metal radionuclide such as ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce; superparamagnetic iron oxide crystals; chromophores and fluorophores having absorption and/or emission maxima in the range 300-1400 nm, especially 600 nm to 1200 nm, in particular 650 to 1000 nm; vesicles containing fluorinated gases (i.e. containing materials in the gas phase at 37° C. which are fluorine containing, eg. SF₆ or perfluorinated C₁₋₆ hydrocarbons or other gases and gas precursors listed in WO97/29783); chelated heavy metal cluster ions (e.g. W or Mo polyoxoanions or the sulphur or mixed oxygen/sulphur analogs); covalently bonded non-metal atoms which are either high atomic number (e.g. iodine) or are radioactive, e.g. ¹²³I, ¹³¹I, etc. atoms; iodinated compound containing vesicles; etc.

Stated generally, the imagable moieties may be (1) a chelatable metal or polyatomic metal-containing ion (i.e. TcO, etc), where the metal is a high atomic number metal (e.g. atomic number greater than 37), a paramagnetic species (e.g. a transition metal or lanthanide), or a radioactive isotope, (2) a covalently bound non-metal species which is an unpaired electron site (e.g. an oxygen or carbon in a persistent free radical), a high atomic number non-metal, or a radioisotope, (3) a polyatomic cluster or crystal containing high atomic number atoms, displaying cooperative magnetic behavior (e.g. superparamagnetism, ferrimagnetism or ferromagnetism) or containing radionuclides, (4) a gas or a gas precursor (i.e. a material or mixture of materials which is gaseous at 37° C.), (5) a chromophore (by which term species which are fluorescent or phosphorescent are included), e.g. an inorganic or organic structure, particularly a complexed metal ion or an organic group having an extensive delocalized electron system, or (6) a structure or group having electrical impedance varying characteristics, e.g. by virtue of an extensive delocalized electron system. Examples of particular imagable moieties are described in more detail below.

Chelated metal imagable moieties: Metal Radionuclides, Paramagnetic metal ions, Fluorescent metal ions, Heavy metal ions and cluster ions. Preferred metal radionuclides include ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu ¹⁹⁹Au, ²⁰³Pb and ¹⁴¹Ce; Preferred paramagnetic metal ions include ions of transition and lanthanide metals (e.g. metals having atomic numbers of 6 to 9, 21-29, 42, 43, 44, or 57-71), in particular ions of Cr, V, Mn, Fe, Co, Ni, Cu, La, Ce, Pr, Nd, Pm, Sm, Eu, Gd, Th, Dy, Ho, Er, Tm, Yb and Lu, especially of Mn, Cr, Fe, Gd and Dy, more especially Gd. Preferred fluorescent metal ions include lanthanides, in particular La, Ce, Pr, Nd, Pm, Sm, Eu, Gd, Th, Dy, Ho, Er, Tm, Yb, and Lu-Eu is especially preferred. Preferred heavy metal-containing imagable moieties may include atoms of Mo, Bi, Si, and W, and in particular may be polyatomic cluster ions (e.g. Bi compounds and W and Mo oxides). The metal ions are desirably chelated by chelant groups in particular linear, macrocyclic, terpyridine and N2S2 chelants, such as for example ethylenediamine-N,N,N′,N′-tetraacetic acid (EDTA); N,N,′,N″,N″-diethylene-triaminepentaacetic Nacid (DTPA); 1,4,7,10-tetraazacyclododecane-N,N′,N″,N′″-tetraacetic acid (DOTA); 1,4,7,10-tetraazacyclododecaneN,N′,N″-triacetic acid (D03A); 1-oxa-4,7,10-triazacyclododecane-N,N′,N″-triacetic acid (OTTA); trans(1,2)-cyclohexanodiethylene-triamine-pentaacetic acid (CDTPA), TMT (terpyridine-bis(methylenaminetetraacetic acid)

Further examples of suitable chelant groups are disclosed in U.S. Pat. No. 4,647,447; U.S. Pat. No. 5,367,080; and U.S. Pat. No. 5,364,613. The imagable moiety may contain one or more such chelant groups, if desired metallated by more than one metal species (e.g. so as to provide the imagable moieties detectable in different imaging modalities). Particularly where the metal is non-radioactive, it is preferred that a polychelant moiety is used.

A chelant or chelating group as referred to herein may comprise the residue of one or more of a wide variety of chelating agents that can complex a metal ion or a polyatomic ion (e.g. TcO).

A chelating agent is a compound containing donor atoms that can combine by coordinate bonding with a metal atom to form a cyclic structure called a chelation complex or chelate. The reside of a suitable chelating agent can be selected from polyphosphates, such as sodium tripolyphosphate and hexametaphosphoric acid; aminocarboxylic acids, such as EDTA (ethylenediaminetetraacetic acid), N-(2-hydroxy)ethylenediaminetriacetic acid, nitrilotriacetic acid, N,N-di(2-hydroxyethyl)glycine, ethylenebis(hydroxyphenylglycine) and diethylenetriamine pentacetic acid; 1,3-diketones, such as acetylacetone, trifluoroacetylacetone, and thenoyltrifluoroacetone; hydroxycarboxylic acids, such as tartaric acid, citric acid, gluconic acid, and 5-sulfosalicyclic acid; polyamines, such as ethylenediamine, diethylenetriamine, triethylenetetraamine, and triaminotriethylamine; aminoalcohols, such as triethanolamine and N-(2-hydroxyethyl)ethylenediamine; aromatic heterocyclic bases, such as 2,21-diimidazole, picoline amine, dipicoline amine and 1,10-phenanthroline; phenols, such as salicylaldehyde, disulfopyrocatechol, and chromotropic acid; aminophenols, such as 8-hydroxyquinoline and oximesulfonic acid; oximes, such as dimethylglyoxime and salicylaldoxime; peptides containing proximal chelating functionality such as polycysteine, polyhistidine, polyaspartic acid, polyglutamic acid, or combinations of such amino acids; Schiff bases, such as disalicylaldehyde 1,2-propylenediimine; tetrapyrroles, such as tetraphenylporphin and phthalocyanine; sulfur compounds, such as toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea; synthetic macrocyclic compounds, such as dibenzo[18-crown-6, (CH₃)₆-[14]-4,11]-diene-N₄, and (2.2.2-cryptate); phosphonic acids, such as nitrilotrimethylene-phosphonic acid, ethylenediaminetetra(methylenephosphonic acid), and hydroxyethylidenediphosphonic acid, or combinations of two or more of the above agents. The residue of a suitable chelating agent preferably comprises a polycarboxylic acid group and preferred examples include: ethylenediamine-N,N,N′,N′-tetraacetic acid (EDTA); N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecaneN,N′,N″-triacetic acid (D03A); 1-oxa-4,7,10-triazacyclododecane-N,N′,N″-triacetic acid (OTTA); trans(1,2)-cyclohexanodiethylene-triamine-pentaacetic acid (CDTPA),other suitable residues of chelating agents comprise proteins modified for the chelation of metals such as technetium and rhenium as described in U.S. Pat. No. 5,078,985, the disclosure of which is hereby incorporated by reference.

Metals can be incorporated into a chelant moiety by any one of three general methods: direct incorporation, template synthesis and/or transmetallation. Direct incorporation is preferred.

It is desirable that the metal ion be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferably in aqueous solution at a pH of between about 5 and about 9, more preferably between pH about 6 to about 8. The chelating agent-containing moiety can be mixed with buffer salts such as citrate, acetate, phosphate and borate to produce the optimum pH. Preferably, the buffer salts are selected so as not to interfere with the subsequent binding of the metal ion to the chelating agent.

Where the imagable moiety contains a single chelant, that chelant may be attached directly to the nanoparticle of the present invention, e.g. via one of the metal coordinating groups of the chelant which may form an ester, amide, thioester or thioamide bond with an amine, thiol or hydroxyl group on the nanoparticle. Alternatively the nanoparticle and chelant may be directly linked via a functionality attached to the chelant backbone, e.g. a CH₂-phenyl-NCS group attached to a ring carbon of DOTA and DTPA as proposed by Meares et al. in JACS 110:6266-6267(1988), or indirectly via a homo or hetero-bifunctional linker, e.g. a bis amine, bis epoxide, diol, diacid, difunctionalized PEG, etc.

### Non-Metal Atomic Imagable Moiety:

Preferred non-metal atomic imagable moieties include radioisotopes such as ¹²³I and ¹³¹I as well as non zero nuclear spin atoms such as ¹⁸F, and heavy atoms such as I. Such imagable moieties, preferably a plurality thereof, e.g. 2 to 200, may be covalently bonded to a linker backbone, either directly using conventional chemical synthesis techniques or via a supporting group, e.g. a triiodophenyl group.

### Organic Chromophoric or Fluorophoric Imagable Moieties:

Preferred organic chromophoric and fluorophoric imagable moieties include groups having an extensive delocalized electron system, e.g. cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes, etc. Examples of suitable organic or metallated organic chromophores may be found in “Topics in Applied Chemistry: Infrared absorbing dyes” Ed. M. Matsuoka, Plenum, N.Y. 1990. Particular examples of chromophores which may be used have absorption maxima between 600 and 1000 nm to avoid interference with haemoglobin absorption. Further such examples include: cyanine dyes: such as heptamethinecyanine dyes. Specific dyes structures useful in the present invention are listed elsewhere in this specification.

### Administration to Human Body or Live Animals:

The contrast agent of the present invention is preferably administered as a pharmaceutical formulation comprising the nanoparticle in a form suitable for administration to a mammal. The administration is suitable for being carried out by injection or infusion of the formulation such as an aqueous solution. The formulation may contain one or more pharmaceutical acceptable additives and/or excipients e.g. buffers; solubilizers such as cyclodextrins; or surfactants such as Pluronic, Tween or phospholipids. Further, stabilizers or antioxidants such as ascorbic acid, gentisic acid or para-aminobenzoic acid and also bulking agents for lyophilisation such as sodium chloride or mannitol may be added.

The present invention also provides a pharmaceutical composition comprising an effective amount (e.g. an amount effective for enhancing image contrast in an in vivo imaging procedure) of a composition of the nanoparticle-based contrast agent of the present invention or a salt thereof, together with one or more pharmaceutically acceptable adjuvants, excipients or diluents.

A further aspect the invention provides the use of a composition of the nanoparticle-based contrast agent of the present invention for the manufacture of a contrast medium for use in a method of diagnosis involving administration of said contrast medium to a human or animal body and generation of an image of at least part of said body.

Still a further aspect of the invention provides a method of generating enhanced images of a human or animal body previously administered with the nanoparticle-based contrast agent composition which method comprises generating an image of at least part of said body.

The core-shell nanoparticles of the present invention may be prepared via surface initiated polymerization. The silica particles may be prepared by the Stober process wherein a tetraorthosilicate is controllably hydrolysed and self-condenses to form particles with silanol groups on the surface. Thermodynamically stable particles may be prepared by condensation of these surface silanol groups on the Stober particles with a monoalkoxysilane. For example, reactive functional groups can be incorporated onto the particle surface to produce a polymerization initiation site. In one embodiment, controlled free radical polymerization initiator such as those for atom transfer radical polymerization (ATRP), nitroxide mediated polymerization (Husseman, M. et al. Macromolecules 1999, 32, 1424-1431) or reversible addition-fragmentation chain transfer polymerization (RAFT) (Li, C. et al. Macromolecules, 2005, 38, 5929) was introduced to the surface of the silica particle. Preferably, atom transfer radical polymerization (ATRP) initiator bromo-isobutyrate was introduced to the surface by reacting with 3-(2-bromoisobutyryloxy)propyldimethyethoxysilane.

The surface functionalization reaction was carried out in an organic solvent such as tetrahydrofuran (THF), methyl ethyl ketone or dioxane under mild heating condition. The initiator particles produced by this process were purified to remove excess silane by precipitating silica nanoparticles in a non-solvent such as hexane or heptane and then centrifuged. The process was repeated several times to remove physically adsorbed initiator on the surface. The silica particle was then redispersed in an organic solvent e.g. toluene, xylene, anisole or methanol or in water, for controlled free radical polymerization reaction: atom transfer radical polymerization.

In another embodiment, reactive functional groups such as amines may be introduced to the surface to induced ring opening polymerization of protected N-carboxyanhydride (NCA) of amino acids to produce poly(amino acid). For example, during the Stober process, primaryl amine groups were introduced to the silica particle surface by addition of trimethoxysilylpropylamine. The polymerization of protected NCA amino acids were carried out in a dry polar solvent such as dimethylamide (DMF). The protecting groups of the amino acids were removed to generate poly(amino acids) with reactive functional groups such as amine or carboxylic acid.

In another embodiment, reactive functional groups such as hydroxy groups may be introduced to the surface to induced ring opening polymerization of cyclic oxide such as ethylene oxide or propylene oxide to produce poly(ethylene oxide) or poly(propylene oxide). For example, during the Stober process, hydroxy groups were introduced to the silica particle surface by addition of silane reagents containing protected hydroxy groups. The polymerization of poly(ethylene oxide) or poly(propylene oxide) can be carried out in a dry solvent such as toluene. The chain end of the polymers can be end capped by functional groups to generate nanoparticles with core-shell nanoparticles with functional groups on the peripheries.

In another embodiment, imaging agents or other useful agents can be incorporated into silica core of the nanoparticle during Stober synthesis. For example, fluorescent or quencher dyes containing reactive alkoxysilane groups were incorporated into the silica core. Surface functionalization to introduce the initiator site for polymerization may be carried out in a similar manner as disclosed above using silica without dye incorporated.

### Synthesis of Quencher/Fluorescent Dye Particle:

The reactive functional groups in polymers shell may be incorporated by polymerization of functional monomers or by modifying the polymer shell with functional groups. The reactive functional groups in the polymer shell include, but are not limited to, thiols, chloromethyl, bromomethyl, amines, carboxylic acid or activated ester, vinylsulfonyls, aldehydes, epoxies, hydrazides, succinimidyl esters, maleimides, a-halo carbonyl moieties (such as iodoacetyls), isocyanates, isothiocyanates, hydroxyl, and aziridines. Preferably the reactive functional group is a thiol, a carboxylic acid, an amine, 4-fluoro-5-nitro-benzoate, or a carboxylic acid activated ester.

The monomers useful for polymerization to form polymer shell include but are not limited to styrenes, (meth)acrylates, and (meth)acrylamide, amino acids, organic cyclic oxide such as ethylene oxide or propylene oxide. The preferred polymerization techniques includes but are not limited to controlled free radical polymerization such as atom transfer radical polymerization (ATRP), ring opening polymerization of ethylene oxide or propylene oxide, ring opening polymerization of N-carboxyanhydride (NCA) of amino acids.

The imaging agents of the present invention are dyes and preferably are near infrared dyes. They contain reactive groups and may be attached to the polymer shell directly or indirectly via the spacer groups. The reactive groups typically are amines, carboxylic acids or their activated esters, 4-fluoro-5-nitro-benzoates, thiols, aldehydes, chloromethyl, and hydroxyls. Preferably, they are amines, carboxylic acids or their activated esters, 4-fluoro-5-nitro-benzoates, thios, and hydroxyls. The spacer groups are enzyme-specific oligopeptides. For example, oligopeptides can be one of the peptides disclosed in International Publication No. WO2004/026344.

The imaging probe of the present invention is activated by cleavage of the imaging agents from the polymer shells by over-expressed enzymes in the disease sites. The imaging agents are attached to the polymer shell via an enzyme specific oliopeptide sequences. Upon activation, some or all imaging agents are released from the nanoparticle of the imaging probe. In the present invention, significant increase of fluorescence upon activation of the probe is detected. Specific enzymes associated with disease are Cathepsion B/H, MMPs, Cathepsin D, prostate specific antigen, and Cathepsin K.

The following examples are provided to illustrate the invention.

The invention has been described in detail with particular reference to certain preferred embodiments thereof, but it will be understood that variations and modifications can be effected within the spirit and scope of the invention.

Materials: Fluorescamine and ethanolamine were purchased from Sigma-Aldrich. Borate buffer was made from boric acid (Sigma, 99%) adjusted by sodium hydroxide. Materials for peptide synthesis are listed in the peptide synthesis section below. Cy7-Q™ was purchased from Amersham (UK), GE. 2-(1H-benzotriazole-1-yl)-1,1,3,3-tetramethyl-uronium hexafluorophosphate (HBTU)/N-hydroxybenzotriazole(HOBt),(ABI, CA), N-Methylpyrrolidone (NMP), Dichloromethane (DCM) and 2.0 M N-Diisopropylethylamine(DIPEA)/NMP were purchased from ABI (CA). N,N-Dimethylformamide (DMF) was purchased from EMD chemicals Inc (Darmstadt, Germany), which was treated with molecular Sieve (EM Science, Gibbstown, N.J.). MMP-2 activated enzyme was purchased from EMD Biosciences (Calbiochem, Calif.). MMP-2 activity assay buffer was purchased from Anaspec, Inc (San Jose, Calif.). PBS buffer (137 mm NaCl, 2.7 mM KCl, 20 mM Na₂HPO₄, 2 mM NaH₂PO₄) was provided by Eastman Kodak Co. (Rochester, N.Y.). Centriprep® filter tubes with a 30,000 Daton molecule weight cutoff were purchased from Millipore Co. (Bedford, Mass.). All reagents were used as received unless specified otherwise.

### EXAMPLE 1

### A Quencher Dye Synthesis

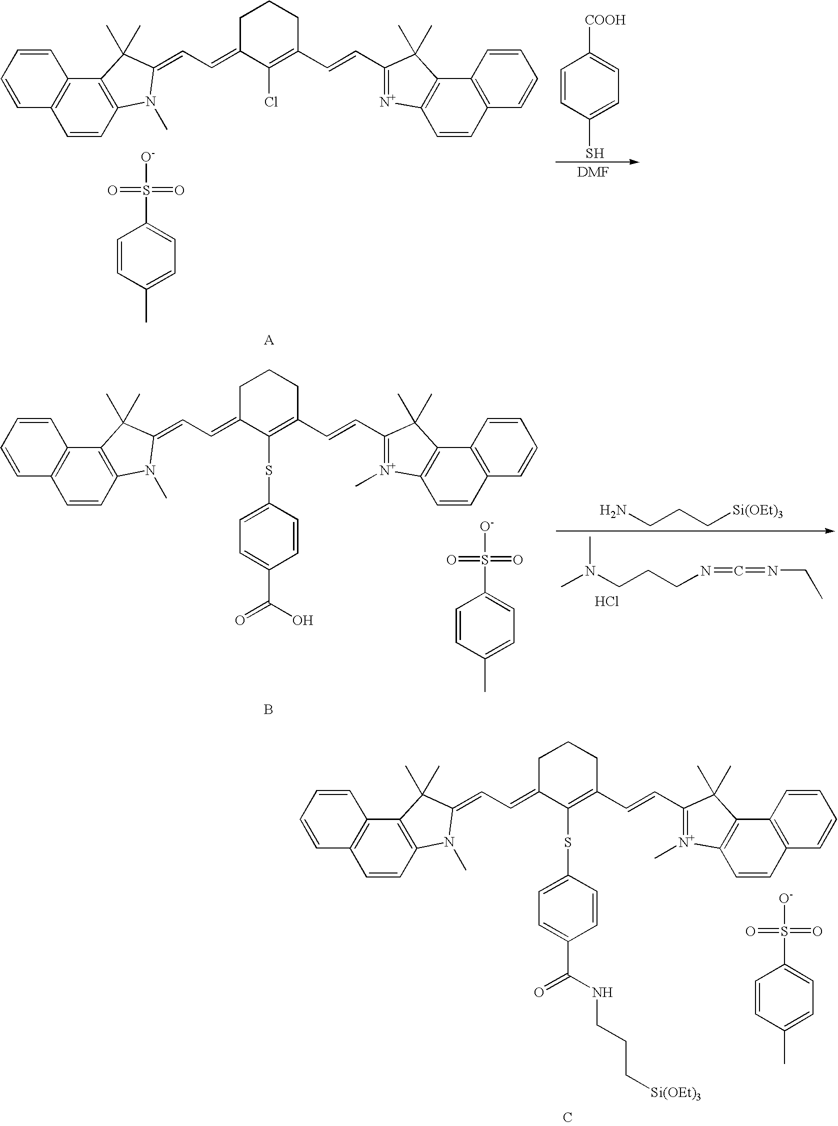

The dye precursor A (6.3 g, 8.3 mmol) and 4-mercaptobenzoic acid (1.54 g, 10 mmol) were dissolved in DMF (60 ml). The mixture was stirred at room temperature under N₂ and the reaction was monitored by both TLC and mass spectroscopy. After 7 hours, the mixture was poured to ether (1 liter), the precipitate was collected, and pure enough for the next step reaction without further purification. 6 grams of B was obtained.

To a solution of compound B (4.4 g, 5 mmol) dissolved in dry pyridine (20 ml) were added 3-aminopropyltriethoxysilane (2.2 g, 10 mmol) and 1-(3-dimethylaminopropyl)3-theylcabodiimide hydrochloride (2 g, 10.4 mmol). The resulting mixture was stirred under N2 at room temperature for 6 hours (monitored by both TLC and mass spectroscopy). The mixture was then poured to ether (200 ml); the dye was precipitated out as sticky solid. The residue after ether being decanted was taken up in dichloromethane and purified through a silica gel column using a mixture of heptane and ethyl acetate (1:1) as eluting solvents. The green band (2.4 g) was collected; a dark green solid was obtained after the solvent removal. Both NMR and mass spectroscopy results agree with the proposed dye structure C.

### EXAMPLE 2

### Synthesis of Quencher Dye Compound C (Example 1) Incorporated Silica Particle

Compound C (4 mg) was dissolved in 200 mL of ethanol. The solution was heated to 55° C., and tetraethoxysilane (7.6 mL), ammonium hydroxyide aqueous solution (28% in water, 6.4 mL) and water (12 mL) were added. The reaction was heated for 4 hours at 55° C. The reaction was cooled down and excess ammonium hydroxide was removed under reduced pressure using rotoevaporation. Particle size was measured by dynamic light scattering to be 30 nm.

### EXAMPLE 3

### Synthesis of Fluorescent Dye Incorporated Silica Particle and Functionalized with Aminotriethoxypropylsilane

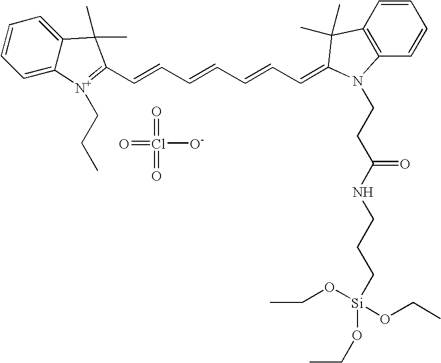

Near infrared dye (4 mg) was dissolved in 200 mL of ethanol. The solution was heated to 55° C., and tetraethoxysilane (7.6 mL), ammonium hydroxyide aqueous solution (28% in water, 2 mL) and water (12 mL) were added. The reaction was heated for 4 hours at 55° C. Particle size was measured by dynamic light scattering to be 14 nm.

The above dyed-particle was further reacted with additional tetraethoxysilane (0.1 mL), aminotriethoxypropylsilane (0.32 g), and hydroxyide aqueous solution (28% in water, 0.06 mL) at 55° C. for 3 hours. After cooling down, excess ammonium hydroxide was removed under reduced pressure. DMF (50 mL) was added to the dyed-particle solution and the solvent was reduced to 30 mL under reduced pressure. NMR study was used to determine the amount of amine groups attached to the surface of the particle.

### EXAMPLE 4

### Synthesis of Core-Shell Nanoparticle with Fluorescent Dye Incorporated Silica Core and Polylysine in Polymer Shell

1. Synthesis of N-Carboxyanhydride (NCA) of Protected Lysine (See Daly, W I H. et al. Tetrahedron Lett. 1988, 29, 5859-5862)

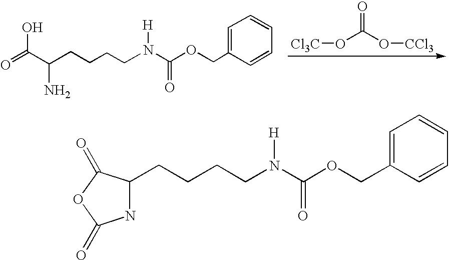

Starting material (10 g, 0.036) was suspended in dry THF 100 mL and triphosgene (4.2 g, 0.015 mol) was added with 5 mL of THF. The reaction became very thick and 100 mL more of THF was added. The reaction was heated to 55° C. for 3 hours and cooled down. The solvent was reduced and the slurry was poured into 300 mL of heptane and cooled down in a freezer. The off-white solid was filtered off and redissoved in THF and then poured into hexane. The solid was filtered off to give 9.4 g (86% yield) of product as white solid.

### 2. Polymerization of NCA of Protected Lysine

To the amine-functionalized dyed-nanoparticle of example 3 in 30 mL of DMF was added 1.9 g of NCA of protected lysine. The reaction was stirred at 5° C. for 7 days. The solution was poured into water and the blue precipitate was filtered off to give 0.92 g of dark blue solid. The amine was then released by deprotecting with trifluoroacetic acid.

### EXAMPLE 5

### Synthesis of Core-Shell Nanoparticle with Silica Core and PEG and Amine Groups in Polymer Shell

### 1. Synthesis of Silane Initiator A

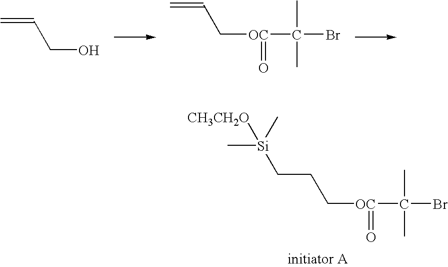

The synthesis of above atom transfer radical polymerization (ATRP) initiator was described in a publication (J. Am Chem. Soc. 2001, 123, 7497-7505).

### 2. Synthesis of Nanoparticle Initiator

Silica nanoparticle (Nissan Chemical, 10-15 nm in MEK, 30 wt % solid) (40 g of 30 wt % solid) was added to a flask and initiator A (14 g) was added. The reaction was heated to 80° C. overnight. The reaction was cooled down and pentane 200 mL was added. The off-white precipitated was filtered off and redissoved in MEK, sonicated for 5 mins and pentane was added to form precipitate. The process was repeated total 5 times to remove any adsorbed initiator. The off-white solid was then dispersed in acetone at about 20 wt % solid.

### 3. Synthesis of Monomer B

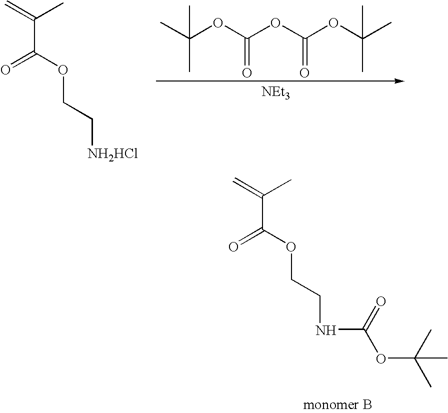

Starting material (85.3 g, 0.52 mol) was suspended in mtheylene chloride (500 mL), and triethylamine (114.7 g, 1.13 mol) was added. The reaction became clear and was cooled to 0° C. t-Boc anhydride (134.9 g, 0.62 mol) was added with 100 mL of methylene chloride. White precipitate formed upon addition. The reaction was stirred at room temperature overnight. The reaction was washed with sodium bicarbonate solution, 1 N HCl and brine and dried over magnesium sulfate. Solvent was reduced and heptane was added until large amount of white precipitate formed. The pure product was obtained as white solid at 92 g (78% yield).

### 4. Polymerization of PEG-Methacrylate and Monomer B on Nanoparticle Initiator

Nanoparticle initiator (0.5 g after removing acetone), PEG-methacrylate (MW 475, 1.2 g), monomer B (1.2 g), anisole 10 mL and 1,1,4,7,10,10-Hexamethyltriethylenetetramine (16 micro liter) were mixed in a round bottomed flask and bubbled with nitrogen for 15 min and CuBr (8 mg) was added quickly. The reaction was heated to 110° C. overnight. The reaction was diluted with THF and poured into 100 mL of hexane. The stickly solid was dried to give core-shell nanoparticle 1.8 g. The molecular weight of the attached polymer shell was determined by dissolving the silica core with dilute HF solution.

5. Deprotection of tBoc to Release Amine Groups

The above core-shell nanoparticle 1.7 g was dissolved in methylene chloride 30 mL and 8 mL of trifluoroacetic acid was added. The reaction was stirred at room temperature overnight. Solvent was removed and the sticky solid was dried under vacuum. It dissolved in methanol and water.

### 6. Sizing of Nanoparticles.

Particle sizing was measured on a Zetasizer based on dynamic light scattering or quasi elastic light scattering (Malvern Instruments, UK) at 25° C. by diluting the concentrated sample 40 times by PBS buffer (PH value: 7.4) and sonicating the solution for 1 minute.

### EXAMPLE 6

### Characterization of Nanoparticles

Analysis of the number of amine groups on nanoparticle surface. The number of primary amines on the surface of nanoparticles was analyzed according to the following procedure.

Fluorescamine was dissolved in DMF at 1 mg/mL. Ethanolamine of 25 μL was dissolved in 975 μL of 0.1M borate buffer of pH 9.0 as standard solution. Dilute this standard at 1:20 by taking 50 μL of above standard and adding 950 μL of borate buffer. From this dilution, prepared the following standard solutions with vigorous stirring.

Standard solution 1 contains 5 μL of diluted ethanolamine, 4945 μL of borate buffer, and then add 50 μL of 1 mg/mL of fluorescamine

Standard solution 2 contains 10 μL of diluted ethanolamine, 4940 μL of Borate buffer, and then add 50 μL of 1 mg/mL of fluorescamine

Standard solution 3 contains 25 μL of diluted ethanolamine, 4925 μL of Borate buffer, and then add 50 μL of 1 mg/mL of fluorescamine

Standard solution 4 contains 50 μL of diluted ethanolamine, 4900 μL of borate buffer, and then add 50 μL of 1 mg/mL of fluorescamine

Sample solutions were prepared according to the following procedures: Sample 1 solution was prepared by mixing 25 μL of 1:100 dilution of nanoparticle of Example 4 5925 μL of borate buffer, and 50 μL of 1 mg/mL of fluorescamine.

Sample 2 was prepared by mixing 50 μL of 1:100 dilution of nanoparticle of Example 4, 5925 μL of borate buffer, and then add 50 μL of 1 mg/mL of fluorescamine.

Fluorescence of standards and samples were measured by using a 1 cm cell at an excitation wavelength of 395 nm, an emission wavelength of 480 nm, 1 second integration and 1 mm slit width.

The primary amine density of the sample is calculated based on the fluorescence of standard and sample solutions, which turns out to be 0.2 mmol/gram particle.

### EXAMPLE 7

### Peptide Synthesis

MMP-2 specific peptide substrates were synthesized on an ABI 433A synthesizer (ABI, CA) by Fmoc chemistry using 2-(1H-benzotriazole-1-yl)-1,1,3,3-tetramethyl-uronium hexafluorophosphate (HBTU)/N-hydroxybenzotriazole(HOBt) (ABI, CA) as the activation agent and Piperidine (ABI) as the deprotection agent. N-Methylpyrrolidone (NMP), Dichloroform (DCM) and 2.0 M N-Diisopropylethylamine(DIPEA)/NMP were also purchased from ABI. N-Fmoc-amido-dPEG4TM-acid was purchased from Quanta Biodesign, Ltd (Powell, Ohio), PEG-polystyrene resin was obtained from ABI. Fmoc-Ahx-OH, Fmoc-Gly-OH, Fmoc-Leu-OH, Fmoc-Val-OH Fmoc-Pro-OH, Fmoc-Arg(Pbf)-OH Fmoc-Ahx-OH were purchased from Anaspec, Inc (CA). The Fmoc-PEG-OH was manually loaded using a double coupling step. Three amino acids were synthesized by solid phase assembly in accordance with the teachings of International Publication No. WO 2004/026344. Thereafter, preactivated Cy7 dye from GE (using HBTU/HOBt/DIPEA) was loaded onto the N-terminal of the peptide on resin by triple coupling. Then, a cocktail of 90% trifluoroacetic acid (Sigma-Aldrich)/5% Triisopropylsilane (Sigma-Aldrich)/5% water was used to deprotect and cleave the peptides. After deprotection, the cocktail solution was filtered via a centrifuge column with 0.2 μm pore size (VectaSpin Micro, Anopore™, Whatman International, Inc, Maidstone England) at 5000 rpm. The filtration was next poured into tert-Butyl methyl ether (anhydrous) 99.8% (Sigma) and washed with tert-Butyl methyl ether three times and finally dried at a reduced vacuum at r.t. The mass value of the unloaded peptide (without dye) was characterized with reverse phase HPLC and MALDI-MS. According to the LC-MS results, the three crude peptide-dye conjugates have the purity of 1: 97.4%, 2. 69.9% and 3. 88% according to total weight area % for the product. Dye conjugated sequence 2 peptide was further purified via reverse phase-HPLC, while the other two peptides were used directly without further purification.

### EXAMPLE 8

### Conjugation of Peptide-Dye Conjugates onto Nanoparticles

### A. Self-Quenching Based Nanoprobes.

Dye conjugated peptide was dissolved in DMF at a concentration of 1 mg/ml. The peptide was then activated by using a 1.2 molar ratio of 0.45 M HBTU/HOBt (relative to peptide molecules) for 5 minutes under vortex, followed by addition of 4 molar ratio of 2M DIPEA into the peptide solution. The solution was kept under shaking for 15 minutes. Nanoparticles of Example 4 were dispersed in DMF at 100 mg/mL. Then the activated peptide-dye conjugate solution was mixed with 40 μL of nanoparticle solution and the mixture was kept under constant shaking overnight. Finally the resultant solution was dropwisely added into large amount of PBS buffer of 7.4. A centriprep® tube was used to remove non-conjugated peptide-dye conjugate and organic solvent by supplementing fresh PBS buffer to the nanoparticle solution until the final filtration solution is colorless. The solution can be further concentrated by centrifugation to remove part of PBS buffer. The solution was then kept frozen for later enzymatic analysis.

### B. FRET Based Nanoprobes.

The FRET based nanoprobes were synthesized by the following procedures. First, 0.05 mg, 0.10 mg, and 0.20 mg of activated Cy7-QTM (purchased from GE) were added into 3 vials of 40 μL of nanoparticle DMF solution of 100 mg/ml, respectively. The reactions were kept overnight. Second, 0.6 mg of activated peptide-dye conjugate (Cy7AhxPLGVRGEE) was added into each of the three above vials and the reaction was kept overnight again. Finally, same purification steps were taken as the preparation of self-quenching probes.

### C. Control Sample: Dye Conjugated Nanoparticles.

The control particle was prepared through the same procedure as above, by mixing 0.5 mg of preactivated Cy7 dye with 40 μL of 100 mg/mL nanoparticles.

### D. The Determination of Conjugation Yield.

Absorbance and fluorescence curves of a series of known concentration peptide-dye conjugate solutions were used to determine the peptide-dye conjugate concentration of filtration solution. The conjugated peptide-dye fraction was determined by the formula: 1-weight of peptide-dye conjugate of filtration solution/total input peptide-dye conjugate. The conjugated primary amine density was calculated as the molar ratio of conjugated peptide-dye conjugate to the total amine number of input silicon nanoparticles.

### EXAMPLE 9

### 96-Well Plate Assay of Specificity of Activatable Nanoparticles

Concentrated nanoparticle solutions from centriprep® centrifugation were diluted by adding certain amount of MMP-2 assay buffer solution (Anaspec, CA); then 100 μL of this nanoparticle assay buffer solution was added into each well and followed by certain amount of enzyme. The solution was then kept under room temperature. The NIR images of these wells were recorded at various intervals using a Kodak imaging station with a 720 nm excitation filter and a 790 nm emission filter. NIR images of control samples without enzyme digestion were also recorded as reference.

### EXAMPLE 10

### Activation of Imaging Probe by Enzyme MMP-2: Spectrometric Assay of Specificity of Activatable Nanopartciles

Spex Fluorolog (1680 0.22 mm Double spectrometer) fluorimeter was used to run enzymatic assay on nanoparticles at a 3 mm slit width using a 1 mm diamond cell. The excitation filter was 740 nm/750 nm and emission filter was 763 mn/775 nm. Typically, 60 μL of concentrated nanoparticle was mixed with 120 μL of MMP assay buffer. An aliquot of 0.2 μg of MMP-2 enzyme (EMD Bioscience) was added and mixed; the resulting solution was then injected into a 1 mm cell. The fluorescence intensity of a same concentration nanoparticle solution without enzyme was used as the starting point of florescence intensity. After a certain time of incubation of nanoparticle with enzyme, the fluorescence intensity or a spectrometric curve was recorded. To reach the full potential of the activatable probe, more batches (Every batch amount is 0.2 μg) of enzyme were added after the leveling off of the enzyme activity. To determine whether primarily it is the cleaved Cy7-peptide fragment or the less quenched nanoparticle which contributes to the fluorescence intensity increase after the incubation of the activatable probes with MMP-2, centriprep® was used to remove the Cy7-peptide fragment from the residual particle; then the fluorescence spectrometric curve of the filtrate and the residual particle were both recorded.

### EXAMPLE 11

### Activation of Imaging Probe by Caner Cell Induced Over-Expression of MMP-2

MCF-7 cells and Fibroblast were purchased from American Type Culture Collection.

MCF-7 cells were grown until 70-80% confluent on tissue culture treated glass slide (BD Biosciences) containing DMEM and 10% FBS and antibiotics as described as the manufacture instruction. The MCF-7 cells were washed with serum free medium for three times and were incubated with Peptide-dye conjugate loaded nanoparticle. The Peptide-dye conjugate loaded nanoparticle was diluted 40 times with serum free medium. 4 hours later, the cells were washed out with serum free medium, then Fluorescence and NIR images were taken under the Olympus BX40 microscope using a MicroFire™ Monochrome Digital Camera (Model S99809) and Monochrome QICAM-IR CCD Digital Camera, respectively.

It was further confirmed that this probe also could detect MMP2 activation by different inducers in cell. In this test, Detroit 548 fibroblast cells were seeded on slide chamber wells, culture until sub-confluence. 2000 of MCF-7 cells were seeded over the fibroblast cells in each well or were incubated with or without thrombin, and were incubated for three days. After washing with serum free medium, the cells were incubated with activatable imaging probe at 0.6 mg/mL concentration, while as control the cells were incubated with NIR labeled nanoparticle (without peptide spacer groups) for 4 hours at a temperature of 37° C. Then Fluorescence and Contrast images were taken under the Olympus BX40 microscope.

1. A nanoparticulate imaging probe comprising an oxide core, a biocompatible polymeric shell covalently attached to the oxide core, a dye that produces emissions in response to electromagnetic radiation, and a cleavable spacer that covalently binds the dye to the probe such that the dye is liberated from the probe when the spacer is cleaved, wherein the probe has a size of less than 100 nm and the emissions of the dye is quenched when the dye is bound to the probe and not quenched when the dye is liberated from the probe.

2. The imaging probe as recited in claim 1, wherein the spacer is comprised of a polypeptide.

3. The imaging probe as recited in claim 1, wherein the oxide core is comprised of an oxide of an element selected from the group consisting of silicon, aluminum, iron, zinc, and zirconium.

4. The imaging probe as recited in claim 1, wherein the oxide core is comprised of an oxide of silicon.

5. The imaging probe as recited in claim 1, wherein the polymeric shell is comprised of a plurality of poly(ethylene glycol) segments.

6. The imaging probe as recited in claim 1, wherein the electromagnetic radiation is infrared radiation.

7. The imaging probe as recited in claim 1, wherein the dye is bound to the cleavable peptide through a functional group selected from the group consisting of an amine, a carboxylic acid, an activated ester, a 4-fluoro-5-nitro-benzoate, a thiol, and a hydroxyl.

8. The imaging probe as recited in claim 7, further comprising an agent covalently bound to the probe wherein the agent is selected from the group consisting of a therapeutic agent, a targeting agent, and a diagnostic agent.

9. The imaging probe as recited in claim 1, wherein the cleavable peptide is bound to the oxide core.

10. The imaging probe as recited in claim 1, wherein the cleavable peptide is bound to the polymeric shell.

11. A nanoparticulate imaging probe comprising an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

12. The imaging probe as recited in claim 11, wherein the oxide core is comprised of an oxide of an element selected from the group consisting of silicon, aluminum, iron, zinc, and zirconium.

13. The imaging probe as recited in claim 11, wherein the oxide core is comprised of an oxide of silicon.

14. The imaging probe as recited in claim 11, wherein the polymeric shell is comprised of a plurality of poly(ethylene glycol) segments.

15. The imaging probe as recited in claim 11, wherein the component is bound to the cleavable peptide through a functional group selected from the group consisting of an amine, a carboxylic acid, an activated ester, a 4-fluoro-5-nitro-benzoate, a thiol, and a hydroxyl.

16. The imaging probe as recited in claim 15, further comprising an agent covalently bound to the probe wherein the agent is selected from the group consisting of a therapeutic agent, a targeting agent, and a diagnostic agent.

17. The imaging probe as recited in claim 11, wherein the cleavable peptide is bound to the oxide core.

18. The imaging probe as recited in claim 11, wherein the cleavable peptide is bound to the polymeric shell.

19. The imaging probe as recited in claim 11, wherein the component which is bound to the cleavable peptide is the dye and the quencher is not bound to the cleavable peptide.

20. The imaging probe as recited in claim 11, wherein the component which is bound to the cleavable peptide is the quencher and the dye is not bound to the cleavable peptide.

21. A process for in vivo imaging comprising the steps of:
administering a nanoparticulate imaging probe to an animal which has a targeted tissue and a non-targeted tissue, wherein the probe comprises an oxide core, a biocompatible polymeric shell covalently attached to the oxide core, a dye that produces emissions in response to near-infrared electromagnetic radiation, and a cleavable peptide that covalently binds the probe to the dye such that the dye is liberated from the probe when the peptide is cleaved, wherein the probe has a size of less than 100 nm and the emissions of the dye is quenched when the dye is bound to the probe and not quenched when the dye is liberated from the probe;
waiting for the probe to accumulate in the targeted tissue which includes an enzyme, wherein the cleavable peptide is configured to be cleaved by the enzyme;
irradiating the targeted tissue with near-infrared electromagnetic radiation of a wavelength absorbable by the dye, thus producing the emissions; and
detecting the emissions of the liberated dye.

22. The process as recited in claim 21, further comprising the steps of
administering a second nanoparticulate imaging probe to the animal, wherein the second probe comprises a second oxide core, a second biocompatible polymeric shell covalently attached to the second oxide core, a second dye that produces emissions in response to near-infrared electromagnetic radiation, and a second cleavable peptide that covalently binds the second probe to the second dye such that the second dye is liberated from the second probe when the second peptide is cleaved, wherein the second probe has a size of less than 100 nm and the emissions of the second dye is quenched when the second dye is bound to the second probe and not quenched with the second dye is liberated from the second probe;
waiting for the second probe to accumulate in the targeted tissue which includes a second enzyme, wherein the second cleavable peptide is configured to be cleaved by the second enzyme;
irradiating the targeted tissue with electromagnetic radiation of a wavelength absorbable by the second dye, thus producing the emissions; and
detecting the emissions of the second liberated dye.
